SEQUENCE LISTING (30 sequences)

SEQ ID NO: 1 - DNA, 2975 bp, Homo sapiens
gaattttctgcaaggatcatatctaagtgcactttttgctgatacttcatttctagacattgagtctcactctaccccccaggctgaagtgcagtggtgtgatctcggttcactgcaacctccgcctccaggttcaagtgattctcgtacctcagcctcccgagtagctgggattacaggcgcctgccaccatgcctggctgatatttatatttttagtagagatggagtttcaccatgttggccaggctggtctcgaactctggacctcagatcttgtagaaaatttcagctgtagcccttggactagaagctgaaataacagaagctgtgtacgatgcattagggtattgaagaaaattaacttttgaattaaatatttggaatataaggaaataaggaaagttgactgaaaatggggcggaagaaaatacaaatcacacgcataatggatgaaaggaaccgacaggtcacttttacaaagagaaagtttggattaatgaagaaagcctatgaacttagtgtgctctgtgactgtgaaatagcactcatcattttcaacagctctaacaaactgtttcaatatgctagcactgatatggacaaagttcttctcaagtatacagaatataatgaacctcatgaaagcagaaccaactcggatattgttgaggctctgaacaagaaggaacacagagggtgcgacagcccagaccctgatacttcatatgtgctaactccacatacagaagaaaaatataaaaaaattaatgaggaatttgataatatgatgcggaatcataaaatcgcacctggtctgccacctcagaacttttcaatgtctgtcacagttccagtgaccagccccaatgctttgtcctacactaacccagggagttcactggtgtccccatctttggcagccagctcaacgttaacagattcaagcatgctctctccacctcaaaccacattacatagaaatgtgtctcctggagctcctcagagaccaccaagtactggcaatgcaggtgggatgttgagcactacagacctcacagtgccaaatggagctggaagcagtccagtggggaatggatttgtaaactcaagagcttctccaaatttgattggagctactggtgcaaatagcttaggcaaagtcatgcctacaaagtctccccctccaccaggtggtggtaatcttggaatgaacagtaggaaaccagatcttcgagttgtcatccccccttcaagcaagggcatgatgcctccactatcggaggaagaggaattggagttgaacacccaaaggatcagtagttctcaagccactcaacctcttgctaccccagtcgtgtctgtgacaaccccaagcttgcctccgcaaggacttgtgtactcagcaatgccgactgcctacaacactgattattcactgaccagcgctgacctgtcagcccttcaaggcttcaactcgccaggaatgctgtcgctgggacaggtgtcggcctggcagcagcaccacctaggacaagcagccctcagctctcttgttgctggagggcagttatctcagggttccaatttatccattaataccaaccaaaacatcagcatcaagtccgaaccgatttcacctcctcgggatcgtatgaccccatcgggcttccagcagcagcagcagcagcagcagcagcagcagccgccgccaccaccgcagccccagccacaacccccgcagccccagccccgacaggaaatggggcgctcccctgtggacagtctgagcagctctagtagctcctatgatggcagtgatcgggaggatccacggggcgacttccattctccaattgtgcttggccgacccccaaacactgaggacagagaaagcccttctgtaaagcgaatgaggatggacgcgtgggtgacctaaggcttccaagctgatgtttgtacttttgtgttactgcagtgacctgccctacatatctaaatcggtaaataaggacatgagttaaatatatttatatgtacatacatatatatatccctttacatatatatgtatgtgggtgtgagtgtgtgtgtatgtgtgggtgtgtgttacatacacagaatcaggcacttacctgcaaactccttgtaggtctgcagatgtgtgtcccatggcagacaaagcaccctgtaggcacagacaagtctggcacttccttggactacttgtttcgtaaagataaccagtttttgcagagaaacgtgtacccatatataattctcccacactagcttgcagaaacctagagggccccctacttgttttatttaactgtgcagtgactgtagttacttaagagaaaatgctttgtagaacagagcagtagaaaagcaggaaccaagaaagcaatactgtacataaaatgtcatttatattttccaacctggcatgggtgtctgttgcaaaggggtgcatgggaaagggctgttgatattaaaaacaaacaaaacaaaaaagccccacacataactgttttgcacgtgcaaaaatgtattgggtcaagaagtgatctttagctaataaagaaagagaatagaaaacacgcatgagatattcagaaaatactagcctagaaatatagagcattaacaaaggaaaattaatatattaagttataattggaatatgtcagaagtttctttttacattcatatcttaaaaattaaagaaactgattttagctcatgtatattttatatgaaagaaaacacccttatgaattgatgactatatataaaattatattcactacttttgaacacattctgctatgaattatttatataagccaaagctatatgttgtaacttttttttagagaatagctttatcttggtttaactctttagttttattttaagaggggaaaacaaaaatatcttgcaagcagaaccttgaaaaaaaaaaaggaattc

SEQ ID NO: 2 - protein, 507 aa, Homo sapiens
MGRKKIQITRIMDERNRQVTFTKRKFGLMKKAYELSVLCDCEIALIIFNSSNKLFQYASTDMDKVLLKYTEYNEPHESRTNSDIVEALNKKEHRGCDSPDPDTSYVLTPHTEEKYKKINEEFDNMMRNHKIAPGLPPQNFSMSVTVPVTSPNALSYTNPGSSLVSPSLAASSTLTDSSMLSPPQTTLHRNVSPGAPQRPPSTGNAGGMLSTTDLTVPNGAGSSPVGNGFVNSRASPNLIGATGANSLGKVMPTKSPPPPGGGNLGMNSRKPDLRVVIPPSSKGMMPPLSEEEELELNTQRISSSQATQPLATPVVSVTTPSLPPQGLVYSAMPTAYNTDYSLTSADLSALQGFNSPGMLSLGQVSAWQQHHLGQAALSSLVAGGQLSQGSNLSINTNQNISIKSEPISPPRDRMTPSGFQQQQQQQQQQQPPPPPQPQPQPPQPQPRQEMGRSPVDSLSSSSSSYDGSDREDPRGDFHSPIVLGRPPNTEDRESPSVKRMRMDAWVT

SEQ ID NO: 3 - protein, 500 aa, Homo sapiens
MGRKKIQITRIMDERNRQVTFTKRKFGLMKKAYELSVLCDCEIALIIFNSSNKLFQYASTDMDKVLLKYTEYNEPHESRTNSDIVEALNKKEHRGCDSPDPDTSYVLTPHTEEKYKKINEEFDNMMRNHKIAPGLPPQNFSMSVTVPVTSPNALSYTNPGSSLVSPSLAASSTLTDSSMLSPPQTTLHRNVSPGAPQRPPSTGNAGGMLSTTDLTVPNGAGSSPVGNGFVNSRASPNLIGATGANSLGKVMPTKSPPPPGGGNLGMNSRKPDLRVVIPPSSKGMMPPLSEEEELELNTQRISSSQATQPLATPVVSVTTPSLPPQGLVYSAMPTAYNTDYSLTSADLSALQGFNSPGMLSLGQVSAWQQHHLGQAALSSLVAGGQLSQGSNLSINTNQNISIKSEPISPPRDRMTPSGFQQQQQQQQQQQPPPPPQPQPRQEMGRSPVDSLSSSSSSYDGSDREDPRGDFHSPIVLGRPPNTEDRESPSVKRMRMDAWVT

SEQ ID NO: 4 - protein, 501 aa, Homo sapiens
MGRKKIQITRIMDERNRQVTFTKRKFGLMKKAYELSVLCDCEIALIIFNSSNKLFQYASTDMDKVLLKYTEYNEPHESRTNSDIVEALNKKEHRGCDSPDPDTSYVLTPHTEEKYKKINEEFDNMMRNHKIAPGLPPQNFSMSVTVPVTSPNALSYTNPGSSLVSPSLAASSTLTDSSMLSPPQTTLHRNVSPGAPQRPPSTGNAGGMLSTTDLTVPNGAGSSPVGNGFVNSRASPNLIGATGANSLGKVMPTKSPPPPGGGNLGMNSRKPDLRVVIPPSSKGMMPPLSEEEELELNTQRISSSQATQPLATPVVSVTTPSLPPQGLVYSAMPTAYNTDYSLTSADLSALQGFNSPGMLSLGQVSAWQQHHLGQAALSSLVAGGQLSQGSNLSINTNQNISIKSEPISPPRDRMTPSGFQQQQQPPPPPQPQPQPPQPQPRQEMGRSPVDSLSSSSSSYDGSDREDPRGDFHSPIVLGRPPNTEDRESPSVKRMRMDAWVT

SEQ ID NO: 5 - protein, 502 aa, Homo sapiens
MGRKKIQITRIMDERNRQVTFTKRKFGLMKKAYELSVLCDCEIALIIFNSSNKLFQYASTDMDKVLLKYTEYNEPHESRTNSDIVEALNKKEHRGCDSPDPDTSYVLTPHTEEKYKKINEEFDNMMRNHKIAPGLPPQNFSMSVTVPVTSPNALSYTNPGSSLVSPSLAASSTLTDSSMLSPPQTTLHRNVSPGAPQRPPSTGNAGGMLSTTDLTVPNGAGSSPVGNGFVNSRASPNLIGATGANSLGKVMPTKSPPPPGGGNLGMNSRKPDLRVVIPPSSKGMMPPLSEEEELELNTQRISSSQATQPLATPVVSVTTPSLPPQGLVYSAMPTAYNTDYSLTSADLSALQGFNSPGMLSLGQVSAWQQHHLGQAALSSLVAGGQLSQGSNLSINTNQNISIKSEPISPPRDRMTPSGFQQQQQQPPPPPQPQPQPPQPQPRQEMGRSPVDSLSSSSSSYDGSDREDPRGDFHSPIVLGRPPNTEDRESPSVKRMRMDAWVT

SEQ ID NO: 6 - protein, 507 aa, Homo sapiens
MGRKKIQITRIMDERNRQVTFTKRKFGLMKKAYELSVLCDCEIALIIFNSSNKLFQYASTDMDKVLLKYTEYNEPHESRTNSDIVEALNKKEHRGCDSPDPDTSYVLTPHTEEKYKKINEEFDNMMRNHKIAPGLPPQNFSMSVTVPVTSPNALSYTNPGSSLVSPSLAASSTLTDSSMLSPPQTTLHRNVSPGAPQRPPSTGNAGGMLSTTDLTVPNGAGSSPVGNGFVNSRASPNLIGATGANSLGKVMPTKSPPPPGGGSLGMNSRKPDLRVVIPPSSKGMMPPLSEEEELELNTQRISSSQATQPLATPVVSVTTPSLPPQGLVYSAMPTAYNTDYSLTSADLSALQGFNSPGMLSLGQVSAWQQHHLGQAALSSLVAGGQLSQGSNLSINTNQNISIKSEPISPPRDRMTPSGFQQQQQQQQQQQPPPPPQPQPQPPQPQPRQEMGRSPVDSLSSSSSSYDGSDREDPRGDFHSPIVLGRPPNTEDRESPSVKRMRMDAWVT

SEQ ID NO: 7 - protein, 507 aa, Homo sapiens
MGRKKIQITRIMDERNRQVTFTKRKFGLMKKAYELSVLCDCEIALIIFNSSNKLFQYASTDMDKVLLKYTEYNEPHESRTNSDIVEALNKKEHRGCDSPDPDTSYVLTPHTEEKYKKINEEFDNMMRNHKIAPGLPPQNFSMSVTVPVTSPNALSYTNPGSSLVSPSLAASSTLTDSSMLSPPQTTLHRNVSPGAPQRPPSTGNAGGMLSTTDLTVPNGAGSSPVGNGFVNSRASPNLIGATGANSLGKVMPTKSPPPPGGGNLGMNSRKPDLRVVIPLSSKGMMPPLSEEEELELNTQRISSSQATQPLATPVVSVTTPSLPPQGLVYSAMPTAYNTDYSLTSADLSALQGFNSPGMLSLGQVSAWQQHHLGQAALSSLVAGGQLSQGSNLSINTNQNISIKSEPISPPRDRMTPSGFQQQQQQQQQQQPPPPPQPQPQPPQPQPRQEMGRSPVDSLSSSSSSYDGSDREDPRGDFHSPIVLGRPPNTEDRESPSVKRMRMDAWVT

SEQ ID NO: 8 - protein, 507 aa, Homo sapiens
MGRKKIQITRIMDERNRQVTFTKRKFGLMKKAYELSVLCDCEIALIIFNSSNKLFQYASTDMDKVLLKYTEYNEPHESRTNSDIVEALNKKEHRGCDSPDPDTSYVLTPHTEEKYKKINEEFDNMMRNHKIAPGLPPQNFSMSVTVPVTSPNALSYTNPGSSLVSPSLAASSTLTDSSMLSPPQTTLHRNVSPGAPQRPPSTGNAGGMLSTTDLTVPNGAGSSPVGNGFVNSRASPNLIGATGANSLGKVMPTKSPPPPGGGNLGMNSRKPDLRVVIPPSSKDMMPPLSEEEELELNTQRISSSQATQPLATPVVSVTTPSLPPQGLVYSAMPTAYNTDYSLTSADLSALQGFNSPGMLSLGQVSAWQQHHLGQAALSSLVAGGQLSQGSNLSINTNQNISIKSEPISPPRDRMTPSGFQQQQQQQQQQQPPPPPQPQPQPPQPQPRQEMGRSPVDSLSSSSSSYDGSDREDPRGDFHSPIVLGRPPNTEDRESPSVKRMRMDAWVT

SEQ ID NO: 9 - DNA, 23 bp, Artificial
  Primer
agaagctgtgtacgatgcattag

SEQ ID NO: 10 - DNA, 22 bp, Artificial
  Primer
acccaaccattctgtctatgtt

SEQ ID NO: 11 - DNA, 24 bp, Artificial
  Primer
agattcatcttcagatagcccata

SEQ ID NO: 12 - DNA, 24 bp, Artificial
  Primer
acaagtcattctgacagttaatgc

SEQ ID NO: 13 - DNA, 23 bp, Artificial
  Primer
agttcattccgtctgtgctctct

SEQ ID NO: 14 - DNA, 26 bp, Artificial
  Primer
aagtagaggtaaagtaaaagtactta

SEQ ID NO: 15 - DNA, 26 bp, Artificial
  Primer
taagtacttttactttacctctactt

SEQ ID NO: 16 - DNA, 25 bp, Artificial
  Primer
gcaacaagatgtttggtcaatctct

SEQ ID NO: 17 - DNA, 22 bp, Artificial
  Primer
agtaacttgagttaccttgcca

SEQ ID NO: 18 - DNA, 24 bp, Artificial
  Primer
gaacctgcttatgttaaccaatga

SEQ ID NO: 19 - DNA, 25 bp, Artificial
  Primer
tctctattcagttcacgttcagtta

SEQ ID NO: 20 - DNA, 24 bp, Artificial
  Primer
tgtattagtgaaagtacccttcag

SEQ ID NO: 21 - DNA, 22 bp, Artificial
  Primer
gatactcaaacctgtagtgagt

SEQ ID NO: 22 - DNA, 22 bp, Artificial
  Primer
ggaagctacagattgactatgt

SEQ ID NO: 23 - DNA, 23 bp, Artificial
  Primer
tgtgagtaccaacagtcttagta

SEQ ID NO: 24 - DNA, 23 bp, Artificial
  Primer
ggttagataacaacacgtaagag

SEQ ID NO: 25 - DNA, 22 bp, Artificial
  Primer
tcacatcatcagtgcttcagaa

SEQ ID NO: 26 - DNA, 21 bp, Artificial
  Primer
cacagaagcacacgttgatca

SEQ ID NO: 27 - DNA, 23 bp, Artificial
  Primer
atagattccgtatggaccttcca

SEQ ID NO: 28 - DNA, 24 bp, Artificial
  Primer
aagacagtgtgtaggccaggagtg

SEQ ID NO: 29 - DNA, 22 bp, Artificial
  Primer
tgcagaggtacttgcaagccat

SEQ ID NO: 30 - DNA, 21 bp, Artificial
  Primer
agatatgtagggcaggtcact

(19) United States
(12) Patent Application Publication
(10) Pub. No.: US 2006/0234245 A1
(43) Pub. Date: Oct. 19, 2006

(54) Pathogenic gene for coronary artery disease

(75) Inventors: Qing Wang, Shaker Heights, OH (US); Eric J. Topol, Chagrin Falls, OH (US); Lejin Wang, Cleveland, OH (US); Chun Fan, Cleveland, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 10/927,747

(22) Filed: Aug. 27, 2004

Related U.S. Application Data
(60) Provisional application No. 60/499,116, filed on Aug. 29, 2003.

(51) Int. Cl. C12Q 1/68

(52) U.S. Cl. 435/6

(57) ABSTRACT

A method of determining a person at risk of developing coronary artery disease includes detecting an alteration of at least one of an MEF2A gene, genes regulated by MEF2A transcription factor, or genes that regulate expression of MEF2A transcription factor of the patient. The alteration substantially reduces the transcription activity of the resulting MEF2A transcription factor.

### RELATED APPLICATION

The present application claims priority to U.S. Provisional Application No. 60/499,116 filed Aug. 29, 2003, which is herein incorporated by reference in its entirety.

The work described in this application was supported, at least in part, by grants R01 HL65630 and R01 HL66251 from the National Institutes of Health. The United States government has certain rights in this invention.

### FIELD OF THE INVENTION

The present invention relates to the myocyte enhancer factor 2A (MEF2A) gene and to the use of the MEF2A gene in the diagnosis and treatment of coronary artery disease.

### BACKGROUND OF THE INVENTION

Coronary artery disease (CAD) and its most important complication, acute myocardial infarction (MI), are the leading causes of disability and deaths in the developed world. Each year, more than 700,000 Americans die from CAD/MI, accounting for one of every 5 deaths. Overall, this disease is estimated to affect more than 20 million Americans. The burden of CAD on the U.S. health care system is immense, with direct and indirect costs totaling approximately >$133 billion annually. But despite this remarkable toll on public health, little is known about the genetic basis of the disease. Many risk factors for CAD and MI have been identified, including family history, hypertension, hypercholesterolemia, obesity, smoking, and diabetes. Several genome-wide linkage scans of affected sibpairs have identified four susceptibility loci for CAD and MI, but the specific genes remain to be identified.

A family of transcription factors, the myocyte enhancer factor-2 family (MEF2), are known to play an important role in morphogenesis and myogenesis of skeletal, cardiac, and smooth muscle cells. There are four members of the MEF2 family, referred to as MEF2A, -B, -C, and -D, in vertebrates. MEF2 factors are expressed in all developing muscle cell types, binding a conserved DNA sequence in the control regions of the majority of muscle-specific genes. Of the four mammalian MEF2 genes, three (MEF2A, MEF2B and MEF2C) can be alternatively spliced, which have significant functional differences. These transcription factors share homology in an N-terminal MADS-box and an adjacent motif known as the MEF2 domain. Together, these regions of MEF2 mediate DNA binding, homo- and heterodimerization, and interaction with various cofactors, such as the myogenic bHLH proteins in skeletal muscle. MEF2 binding sites, CT(A/T)₄ TAG/A, are found in the control regions of the majority of skeletal, cardiac, and smooth muscle genes. The C-termini of the MEF2 factors function as transcription activation domains and are subject to complex patterns of alternative splicing. Additionally, biochemical and genetic studies in vertebrate and invertebrate organisms have demonstrated that MEF2 factors regulate myogenesis through combinatorial interactions with other transcription factors.

Loss-of-function studies indicate that MEF2 factors are essential for activation of muscle gene expression during embryogenesis. During mouse embryogenesis, the MEF2 genes are expressed in precursors of cardiac, skeletal and smooth muscle lineages and their expression is maintained in differentiated muscle cells. The MEF2 factors are also expressed at lower levels in a variety of nonmuscle cell types. Targeted inactivation of MEF2C has been shown to result in embryonic death at about E9.5 due to heart failure. In the heart tubes of MEF2C mutant mice, several cardiac genes fail to be expressed, including a-MHC, ANF, and a-cardiac actin, whereas several other cardiac contractile protein genes are expressed normally, despite the fact that they contain essential MEF2 binding sites in their control regions. These results have demonstrated the essential role of MEF2C for cardiac development and suggest that other members of the MEF2 family may have overlapping functions that can support the expression of a subset of muscle genes in the absence of MEF2C. In Drosophila, there is only a single MEF2 gene, called D-MEF2. In embryos lacking D-MEF2, no muscle structural genes are activated in any myogenic lineage, demonstrating that MEF2 is an essential component of the differentiation programs of all muscle cell types.

### SUMMARY OF THE INVENTION

The present invention is directed to a method of identifying a patient that has, or is at risk of developing coronary artery disease (CAD) or a myocardial infarction (MI) by determining if at least one of a myocycte enhancer factor 2A (MEF2A) gene, genes regulated by MEF2A transcription factor, or genes that regulate expression of MEF2A transcription factor of the patient is mutated. Mutations of the MEF2A gene, genes regulated by MEF2A transcription factor, or genes that regulate expression of MEF2A transcription factor can include nucleotide additions, substitutions, or deletions relative to the nucleotide sequence of these genes.

Patients identified by this method can be those that are exhibiting clinical characteristics suggesting that they may have CAD or a MI, or individuals that do not exhibit clinical characteristics or symptoms of CAD and MI. In the first case, the method can be used to make or confirm diagnosis of CAD or MI and, in the latter case, the method can be used to predict whether the patient or their offspring are likely to develop or are vulnerable to the CAD and MI. Identifying those who are vulnerable to CAD and MI is a fundamental strategy to the prevention of CAD and MI. Preventive measures taken by high risk individuals may save their lives.

In general, the likelihood of a patient having or developing CAD and MI is dependent on the particular genetic mutation. Genetic mutations that prevent resulting MEF2A proteins from functioning as a transcription factor can trigger the pathogenesis of CAD and acute MI in a patient. For example, patients having a 21 base-pair deletion in exon 11 of the MEF2A gene were found to have CAD or a MI. The 21 base pair deletion caused a 7 amino acid deletion (Δ7aa) in a resulting MEF2A protein. This Δ7aa prevents MEF2A protein from exerting its function as a transcription factor.

The extent to which the at least one of MEF2A gene, genes regulated by MEF2A transcription factor, or genes that regulate expression of MEF2A transcription factor has been mutated can be determined by any means including direct nucleotide analysis or hybridization under conditions selected to reveal mutations. One preferred method is to amplify one or more regions of the relevant gene using polymerase chain reaction (PCR) and to then analyze the amplification products, for example, by sequence analysis, heteroduplex analysis, or single strand conformational polymorphism analysis. In an aspect of the invention, the region amplified corresponds to one or more of exons 1-11 of the MEF2A gene.

A further aspect of the invention relates to a method of identifying a patient that has, or is likely to develop coronary artery disease (CAD) or myocardial infarction (MI) by determining if an MEF2A protein (i.e., MEF2A transcription factor) is mutated. The mutation to the MEF2A protein can include amino acid additions, substitutions, or deletions that prevent the MEF2A protein from functioning as a transcription factor. Mutations in MEF2A protein can be detected by methods, such as enzyme linked immunosorbent assays (ELISAs), Western blots, immunoprecipitations and immunofluorescence.

Yet another aspect of the invention is to exploit the MEF2A gene and the development of or risk of developing CAD in humans. Thus, it is an aspect of the invention to use MEF2A polypeptides and polynucleotides for treatment and diagnosis of CAD or MI and for identifying compounds that can modulate expression or function of the polypeptides or polynucleotides and are thus useful for treatment and diagnosis of CAD or MI.

The invention also relates to using the polynucleotides and polypeptides to identify compounds that are useful in the treatment and diagnosis of CAD or MI. The compounds can act as agonists or antagonists of MEF2A expression or function. The polynucleotides and polypeptides serve as both a target to identify compounds and may themselves provide a source for derivative compounds that can act as an agonist or antagonist of MEF2A expression or function. The invention is further directed to using these compounds to treat and diagnose CAD. In one embodiment, methods are directed to treating cells, tissues, or animal models associated with the disorder using the MEF2A gene or gene product as a reagent or target for treatment.

The invention is thus also directed to methods of using the MEF2A gene, genes regulated by MEF2A transcription factor, or genes that regulate expression of MEF2A transcription factor as a reagent or target to screen for agents that modulate the levels or effectively reverse the mutation or other abnormality in the MEF2A gene, genes regulated by MEF2A transcription factor, or genes that regulate expression of MEF2A transcription factor. Accordingly, the invention provides methods for identifying agonists and antagonists of the MEF2A gene, genes regulated by MEF2A transcription factor, or genes that regulate expression of MEF2A transcription factor. These agents can be used to diagnose CAD by their effects on the level or function of the MEF2A gene or gene product. By identifying agents that are capable of modulating the expression or function of the MEF2A gene or gene product, methods are thus provided for affecting the development of or course of CAD or MI in an individual by modulating the level or function of the MEF2A gene or gene product. Further, by providing these agents that modulate the expression, methods are provided for assessing the effect of treatment in cell and animal models.

By identifying agents that are capable of interacting with, or otherwise allowing detection of abnormal expression or function of the MEF2A gene or gene product, methods are thus provided for diagnosing the development of, or risk of developing, CAD or MI. This can be in the context of an individual patient, monitoring clinical trials, and assessing MEF2A gene function or efficacy of treatment in cell and animal models. The invention also provides cell and animal model systems for studying CAD and MI based on alterations in the MEF2A gene or gene product in the model.

### BRIEF DESCRIPTION OF THE DRAWINGS

Further features of the present invention will become apparent to those skilled in the art to which the present invention relates from reading the following description of the invention with reference to the accompanying drawings in which:

FIG. 1 illustrates the genetic linkage of coronary artery disease (CAD) and myocardial infarction (MI) to chromosome 15q26. (A) Pedigree structure and genotypic analysis of kindred QW1576. Individuals with characteristic features of CAD and MI (see Table 1) are indicated by closed squares (males) or closed circles (females). Unaffected individuals are indicated by open symbols. Normal males under the age of 50 years or normal females under 55 years are shown with light gray color as uncertain phenotype. Deceased individuals are indicated by a slash, “/”. The proband is indicated by an arrow. Each individual's ID# is shown below each symbol. The results of genotypic analysis are shown below each symbol. Genotypes for markers D15S104, D15S212, D15S120, and D15S87 are shown. Initial linage was identified with D15S120, which yielded a Lod score of 4.19 at a recombination fraction of 0. Haplotype cosegregating with the disease is indicated by a black vertical bar. Haplotype analysis indicates that CAD/MI in kindred QW1576 is linked to markers at chromosome 15q26. (B) Coronary angiogram from the proband of a patient who experienced an inferior MI attributed to this plaque rupture lesion (arrow) with a 70% narrowing in the distal right coronary artery. This lesion is at a bifurcation site typical of the pattern of coronary atherosclerosis. It was stented and follow up angiography of the site demonstrated wide patency, without any renarrowing. (C) Ideogram of chromosome 15 with Geimsa banding pattern and localization of adCAD/MI1 locus. The genetic map with chromosome 15q26 markers and location of the MEF2A gene are shown on the right.

FIG. 2 illustrates the MEF2A intragenic deletion cosegregates with CAD/MI in kindred QW1576. (A) Pedigree showing clinical status (described in FIG. 1 legend) and genetic status: “+” indicates the presence of the 21-bp deletion of MEF2A (heterozygous); “−”, absence of the deletion. (B) DNA sequence analysis of the wild type (WT) allele and the 21-bp deletion allele (Δ21bp) of MEF2A. Sequence analysis of exon 11 of MEF2A in the proband (II.1) revealed the presence of a deletion. The wild type and deletion alleles were separated by a 3% agarose gel or an SSCP (single strand conformation polymorphism) gel, purified and sequenced directly. The location of Δ21bp is indicated. (C) Δ21bp results in a deletion of 7 amino acids of MEF2A (ΔQ₄₄₀P₄₄₁P₄₄₂Q₄₄₃P₄₄₄Q₄₄₅P₄₄₆ or Δ7aa).

FIG. 3 illustrates a functional characterization of wild type and Δ7aa MEF2A proteins by immunofluorescense. (A, B, C) MEF2A deletion Δ7aa causes a sever defect in nuclear localization of MEF2A protein in three cell types (A, human umbilical vascular endothelial cells; B, human aortic smooth muscle cells; C, HeLa cells). Cells were transfected with expression constructs for wild type (WT) and mutant MEF2A proteins tagged with a FLAG-epitope. Immunostaining was then carried out using a mouse anti-FLAG M2 as the primary antibody, and an FITC conjugated sheep anti-mouse IgG as the secondary antibody (green immunostaining signal). The nucleus was stained with DAPI (blue signal). The wild type MEF2A is completely localized into the nucleus, whereas mutant MEF2A protein with Δ7aa is distributed in the cytoplasm in all cells studied. (D) Co-localization of MEF2A and CD31 (PCAM, an endothelial cell specific marker) in the endothelium of human coronary arteries. Cryo-sections (6 μm thick) of human coronary arteries were immunostained with the anti-MEF2A rabbit polyclonal antiserum (MEF2A). The adjacent sections were used for immunostaining with an anti-CD31 monoclonal antibody. The sections were then incubated with the FITC-conjugated anti-rabbit or anti mouse IgG as the secondary antibodies (green signal). Note that the MEF2A expression pattern is almost identical to the CD31 expression pattern L, lumen; E, endothelium.

FIG. 4 illustrates the functional characterization of wild type and Δ7aa MEF2A proteins by transcriptional activation assay. The effect of the 7 amino acid deletion of MEF2A on transcription activation activity was analyzed in the presence or absence of the zinc-finger transcription factor GATA-1. The promoter region, from −700 bp to +1 bp upstream from the transcriptional start site, of ANF was fused to the luciferase gene, and used as the reporter gene (the ANF₋₇₀₀ promoter) for transcriptional activation assay. Transcriptional activity is shown as relative luciferase activity on the y axis. The transcriptional activity of the reporter gene only (vector) was set arbitrarily to 1. WT, wild type MEF2A; Δ7aa, the 7 amino acid deletion of MEF2A; WT/Δ7aa, co-expression of both wild type and mutant MEF2As. Transfections were performed in HeLa cells using LipofectAMINE 2000 (Invitrogen) with 50 ng of DNA for the MEF2A or GATA-1 expression construct, 1 μg of DNA for the reporter gene, and 50 ng of internal control plasmid pSV—galactosidase (for normalizing the transfection efficiency). Western blot analysis and immunostaining showed that both wild type and mutant MEF2A were successfully expressed in transfected HeLa cells (data not shown). The data shown were from two independent experiments in triplicate, and are expressed as mean±S.E.

FIG. 5 illustrates expression of MEF2A protein in proliferating human vascular smooth muscle cells (HVSMC) and human umbilical vacular endothelial cells (HVSMC) and human umbilical vascular endothelial cells (HUVEC). Cultured HVSMC and HVEC were co-immunostained with rabbit polyclonal anti-MEF2A antiserum (Santa Cruz Biotechnology, Santa Cruz, Calif.) and monoclonal anti-actin (Sigma St. Louis, Mo.) as the primary antibodies. The secondary antibodies are the anti-rabbit IgG Cy3-conjugated secondary antibody (Sigma, St. Louis, Mo.) (red signal for MEF2A) and anti-mouse FITC-conjugated secondary antibody (Sigma, St. Louis, Mo.) (green signal for actin). The nuclei were stained with DAPI (blue) and the cytoplasm was stained with monoclonal anti-actin. Note that MEF2A signal co-localizes with DAPI in the nuclei of both HVSMC and HUVEC.

FIG. 6 illustrates DNA sequence analysis of the wild type (WT) allele and and the G to A substituted allele of MEF2A. Sequence analysis of exon 7 of MEF2A in the transcription activation domain revealed the presence of the substitution. The wild type and substituted alleles were separated by a 3% agarose gel or an SSCP (single strand conformation polymorphism) gel, purified and sequenced directly. The location of G to A substitution is indicated. The G to A substitution results in a G283D mutation of MEF2A.

FIG. 7 illustrates DNA sequence analysis of the wild type (WT) allele and and the A to G substituted allele of MEF2A. Sequence analysis of exon 7 of MEF2A in the transcription activation domain revealed the presence of the substitution. The wild type and substituted alleles were separated by a 3% agarose gel or an SSCP (single strand conformation polymorphism) gel, purified and sequenced directly. The location of A to G substitution is indicated. The A to G substitution results in a N263S mutation of MEF2A.

FIG. 8 illustrates DNA sequence analysis of the wild type (WT) allele and and the C to U substituted allele of MEF2A. Sequence analysis of exon 7 of MEF2A in the transcription activation domain revealed the presence of the substitution. The wild type and substituted alleles were separated by a 3% agarose gel or an SSCP (single strand conformation polymorphism) gel, purified and sequenced directly. The location of C to U substitution is indicated. The C to U substitution results in a P279L mutation of MEF2A.

FIG. 9 illustrates (A) a structrue of MEF2A showing CAD/MI-causing mutations; and (B) a graph of transcriptional assays to demonstrate that mutations N263S, P279L, and G283D are functional mutations that disrupt the transcription activity of MEF2A.

FIG. 10 illustrates that since one CAD patient has 0 CAG repeat and three CAD patients have only 4 CAG repeats and normal people do not have (CAG)0 and (CAG)4, (CAG)0 and (CAG)4 repeats may be associated with CAD.

### DETAILED DESCRIPTION OF THE INVENTION

The present invention relates to the discovery that expression of an altered form of myocyte enhanced factor 2A (MEF2A) transcription factor (or protein) is a factor in coronary artery disease (CAD) and myocardial infarction (MI) in humans. Specifically, the inventors have discovered that the occurrence of mutations in the MEF2A gene co-segregates with CAD/MI in a large family. The inventors have also discovered that the molecular signaling pathway(s) mediated by MEF2A, i.e. genes regulated by MEF2A transcription factor, or genes that regulate expression of MEF2A transcription factor, is involved in the development of CAD and MI.

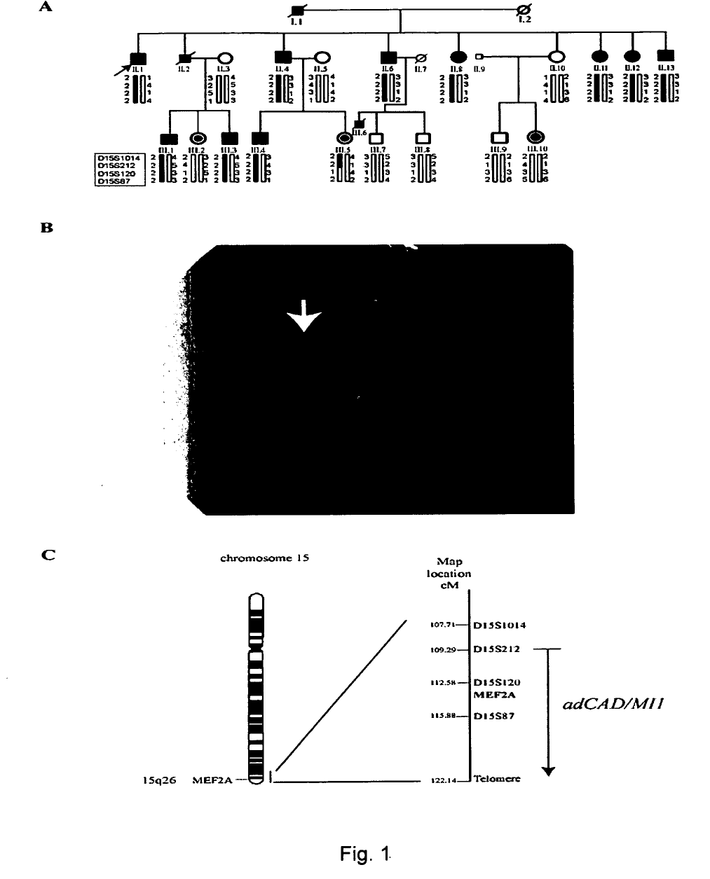

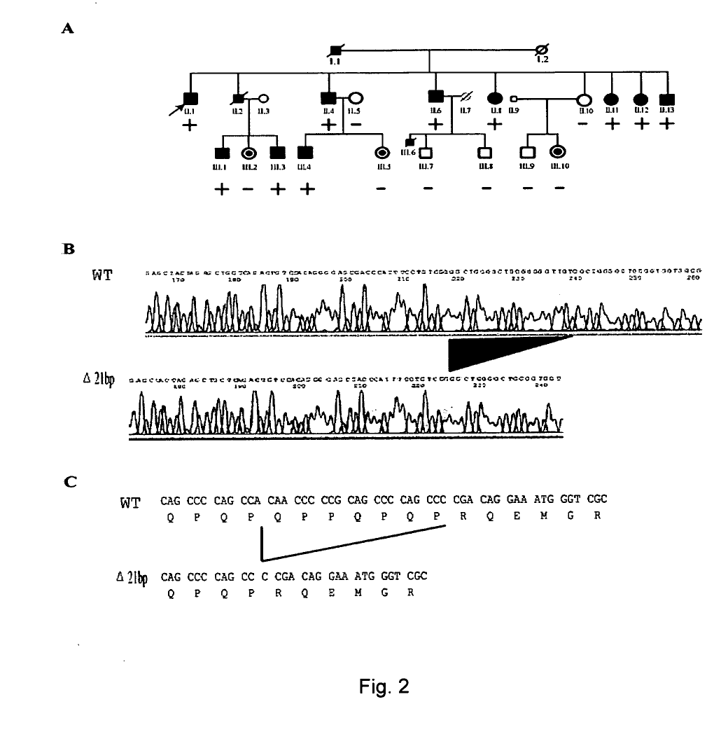

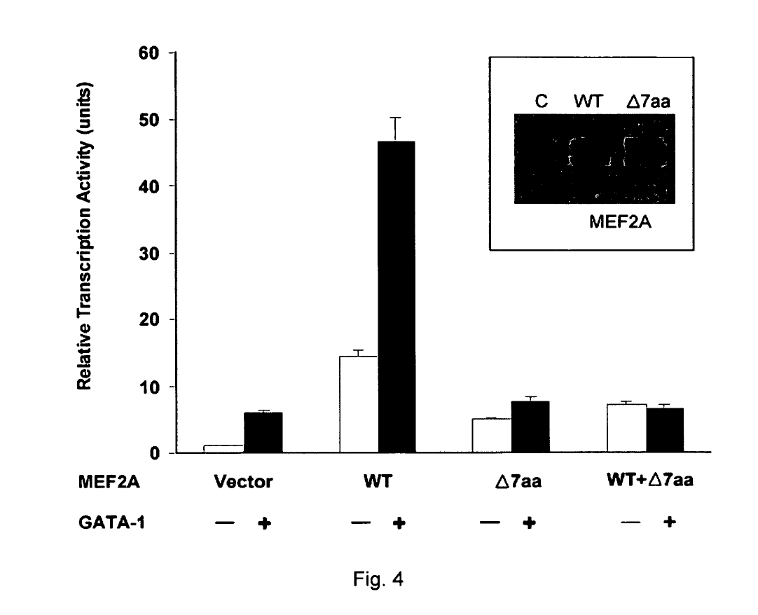

The MEF2A gene, which is located on chromosome 15q26 (FIG. 1), has a wild-type nucleotide sequence that corresponds with SEQ ID NO: 1. The MEF2A gene contains 11 exons and encodes a protein having an amino acid sequence corresponding with SEQ ID NO: 2. In an initial screening of 13 individuals with an autosomal CAD/MI against 119 controls for mutations in the MEF2A gene, the inventors identified a novel 21-base pair deletion in exon 11 of the MEF2A gene (FIGS. 2A-2B) for each of the 13 individuals. The 21-base pair deletion resulted in a deletion of 7 amino acids of the MEF2A protein (ΔQ₄₄₀P₄₄₁P₄₄₂Q₄₄₃P₄₄₄Q₄₄₅P₄₄₆ or Δ7aa) in each of the individuals as illustrated in FIG. 2C. The mutated protein with the Δ7aa had an amino acid sequence that corresponds with SEQ ID NO: 3. The Δ7aa of MEF2A protein was believed to cause conformational change of the MEF2A protein and result in protein trafficking defects. For example, FIG. 4 illustrates the results of transcriptional assays to demonstrate that the Δ7aa mutation is a functional mutation that disrupts the transcription activity of MEF2A. Such a defect prevented MEF2A from exerting its function as a transcription factor and altered the expression profile of MEF2A-target genes. As MEF2A was found to play an important role in cell development and function, and pathogenesis of CAD and MI is associated with endothelial dysfunction, the mutation of the MEF2A was determined to be relevant to CAD and MI. The MEF2A gene 21-bp deletion and the MEF2A protein Δ7aa was absent in the 119 control individuals without CAD/MI.

In addition, exon 11 of the MEF2A gene contains a trinucleotide repeat (11 CAG repeats), which results in 11 contiguous glutamine residues (11Q) at the C-terminus of the MEF2A protein. It was found that (CAG)₁₁ is polymorphic and that normal people only have 8-11 (CAG)s. However, three other independent patients with CAD/MI have no, or only 5 and 6 CAGs (SEQ ID NO: 5 and SEQ ID NO: 6), respectively, which suggests that different variations of the CAGs repeat in MEF2A are associated with CAD and MI.

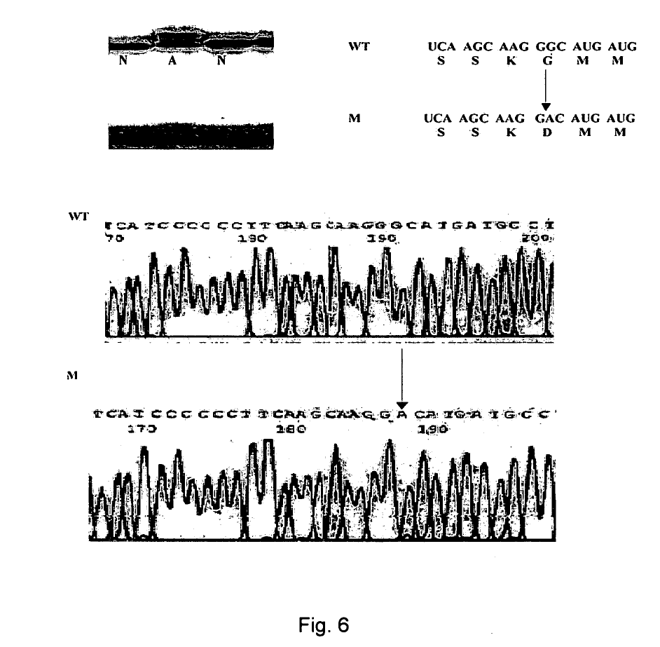

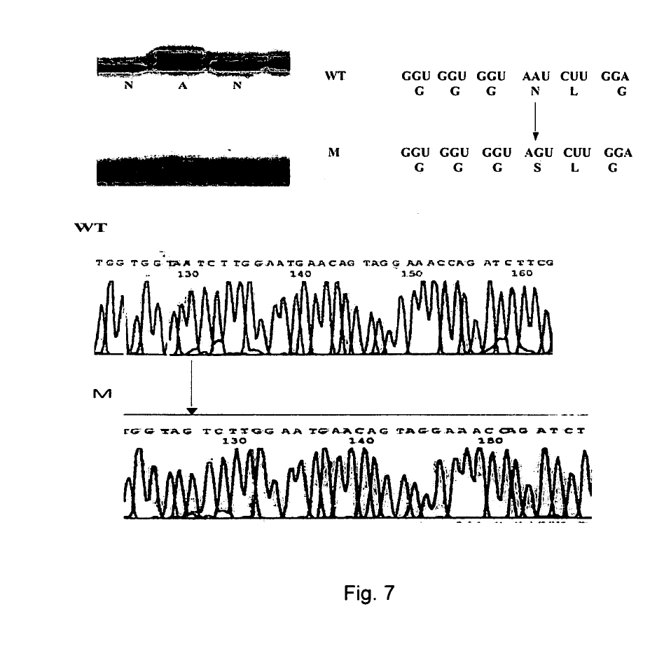

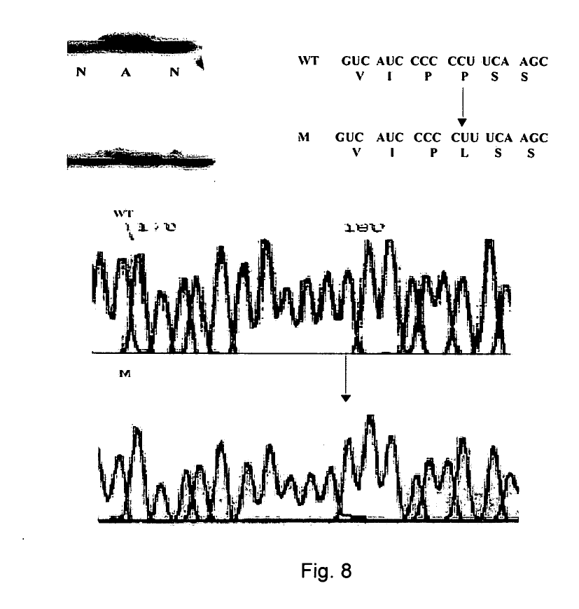

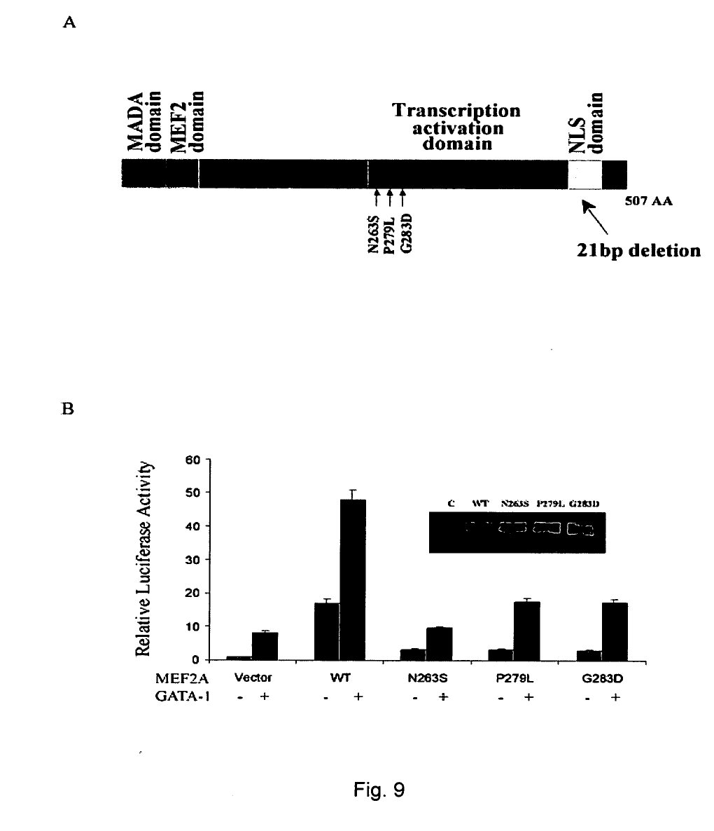

Subsequent to this study, three new mutations (FIGS. 6-8) were identified in exon 7 of the MEF2A gene in four of 200 CAD/MI patients. None of the MEF2A mutations were detected in 200 normal individuals. The resulting MEF2A proteins mutations included N263S in two independent CAD/MI patients, P279L in one patient, and G283D in another patient. The amino sequence for these three MEF2A mutations corresponds respectively with SEQ ID NO: 7, SEQ ID NO: 8, and SEQ ID NO: 9. The three mutations are located close to the major transcription activation domain of MEF2A (amino acids 274-373) and significantly reduced the transcription activity of MEF2A protein, which suggests that N263S, P279L, and G283D are functional mutations that cause CAD and MI. For example, FIG. 9 illustrates the results of transcriptional assays to demonstrate that mutations N263S, P279L, and G283D are functional mutations that disrupt the transcription activity of MEF2A

One aspect of the present invention is therefore directed to methods of using the MEF2A or gene products as a target to detect CAD and MI or the risk of developing CAD and MI. The invention is also directed to methods for determining the molecular basis CAD and MI or the risk of CAD and MI using the MEF2A gene or gene products as a target. It is understood that “gene product” refers to all molecules derived from the gene, especially RNA and protein. cDNA is also encompassed, where, for example, made by naturally-occurring reverse transcriptase.

In an aspect of the invention, the method includes detecting the MEF2A gene itself and/or alterations in copy number, genomic position, and nucleotide sequence of the MEF2A gene. Alterations in the MEF2A nucleotide sequence include the insertion, deletion, point mutation, and inversion of nucleic acids of nucleotide sequece. The alterations can occur at any position within the gene, including coding, noncoding, transcribed, and non-transcribed, regulatory regions. For example, the alteration can be a 21 base pair deletion in exon 11 of the MEF2A gene or point mutations in exon 7 of the MEF2A gene. Other alterations that can be detected include nucleic acid modification, such as methylation, gross rearrangement in the genome such as in a homogeneously-staining region, double minute chromosome or other extrachromosomal element, or cytoskeletal arrangement.

The present invention also encompasses the detection of RNA transcribed from the MEF2A gene. Detection of the RNA transcribed from the MEF2A gene encompasses alterations in copy number and nucleotide sequence. Sequence changes include insertion, deletion, point mutation, inversion, and splicing variation. Detection of MEF2A RNA can be indirectly accomplished by means of its cDNA.

MEF2A DNA and RNA levels and gross rearrangement can be analyzed by any of the standard methods known in the art. In such methods, nucleic acid can be isolated from a cell or analyzed in situ in a cell or tissue sample. For detecting alterations in nucleic acid levels or gross rearrangement, all, or any part, of the nucleic acid molecule can be detected. Nucleic acid reagents derived from any desired region of the MEF2A gene can be used as a probe or primer for these procedures. Copy number can be assessed by in situ hybridization or isolation of nucleic acid from the cell and quantitation by standard hybridization procedures such as Southern or Northern analysis. Genes can be amplified in the forms of homogeneously-staining regions or double minute chromosomes. Accordingly, one method of detection involves assessing the cellular position of an amplified gene. This method encompasses standard in situ hybridization methods, or alternatively, detection of an amplified fragment derived from digestion with an appropriate restriction enzyme recognizing a sequence that is repeated in the amplified unit.

Identifying nucleic acid modifications, such as methylation, can be analyzed by any of the known methods in the art for digesting nucleic acid and analyzing modified nucleotides, such as by BPLC, thin-layer chromatography, mass spectra analysis, and the like. Gross rearrangements in the genome are preferably detected by means of in situ hybridization, although this type of alteration can also be assessed by means of assays involving normal cellular components with which the genes are normally found, such as in specific membrane preparations.

Mutations in MEF2A nucleic acid can be analyzed by any of the standard methods known in the art. Nucleic acid can be isolated from a cell or analyzed in situ in a cell or tissue sample by means of specific hybridization probes designed to allow detection of the mutation. The portion of the nucleic acid that is detected preferably contains the mutation. It is to be understood that in some embodiments, as where the mutation affects secondary structure or other cellular association, distant regions affected by the mutation can be detected. The nucleic acid reagents can be derived from the mutated region of the MEF2A gene to be used as a probe or primer for the procedures. However, as discussed above, nucleic acid reagents useful as probes can be derived from any position in the nucleic acid. RNA or cDNA can be used in the same way.

In certain aspects of the invention, detection of the mutation involves the use of a probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al., Science 241:1077-1080 (1988); and Nakazawa et al., PNAS 91:360-364 (1994)), the latter of which can be particularly useful for detecting point mutations in the gene (see Abravaya et al., Nucleic Acids Res. 23:675-682 (1995)). This method can include the steps of collecting a sample of cells from a patient, isolating nucleic acid (e.g., genomic, mRNA or both) from the cells of the sample, contacting the nucleic acid sample with one or more primers which specifically hybridize to a gene under conditions such that hybridization and amplification of the gene (if present) occurs, and detecting the presence or absence of an amplification product, or detecting the size of the amplification product and comparing the length to a control sample (e.g., a wild-type MEF2A nucleic acid). Deletions and insertions can be detected by a change in size of the amplified product compared to the normal genotype. Point mutations can be identified by hybridizing amplified DNA to normal (or wild-type) RNA or antisense DNA sequences.

Alternatively, mutations in a MEF2A gene can be directly identified, for example, by alterations in restriction enzyme digestion patterns determined by gel electrophoresis. Further, sequence-specific ribozymes can be used to score for the presence of specific mutations by development or loss of a ribozyme cleavage site. Perfectly matched sequences can be distinguished from mismatched sequences by nuclease cleavage digestion assays or by differences in melting temperature. Sequence changes at specific locations can also be assessed by nuclease protection assays such as RNase and SI protection or the chemical cleavage method. Furthermore, sequence differences between a mutant MEF2A gene and a wild-type gene can be determined by direct DNA sequencing. A variety of automated sequencing procedures can be utilized when performing the diagnostic assays ((1995) Biotechniques 19:448), including sequencing by mass spectrometry (e.g., PCT International Publication No. WO 94/16101; Cohen et al., Adv. Chromatogr. 36:127-162 (1996); and Griffin et al., Appl. Biochem. Biotechnol. 38:147-159 (1993)).

Other methods for detecting mutations in the gene include methods in which protection from cleavage agents is used to detect mismatched bases in RNA/RNA or RNA/DNA duplexes (Myers et al., Science 230:1242 (1985)); Cotton et al., PNAS 85:4397 (1988); Saleeba et al., Meth. Enzymol. 217:286-295 (1992)), electrophoretic mobility of mutant and wild type nucleic acid is compared (Orita et al., PNAS 86:2766 (1989); Cotton et al., Mutat. Res. 285:125-144 (1993); and Hayashi et al., Genet. Anal. Tech. Appl. 9:73-79 (1992)), and movement of mutant or wild-type fragments in polyacrylamide gels containing a gradient of denaturant is assayed using denaturing gradient gel electrophoresis (Myers et al., Nature 313:495 (1985)). Examples of other techniques for detecting point mutations include, selective oligonucleotide hybridization, selective amplification, and selective primer extension.

Methods of detection of a mutation of the MEF2A gene can also include detection of the MEF2A protein encoded by the MEF2A gene. Detection encompasses assessing protein levels, mutation, post-translational modification, and subcellular localization. Mutations encompass deletion, insertion, substitution and inversion. Mutations at RNA splice junctions can result in protein splice variants.

MEF2A protein levels can be analyzed by any of the standard methods known in the art. MEF2A protein can be isolated from the cell or analyzed in situ in a cell or tissue sample. Quantification of the MEF2A protein can be accomplished in situ, for example by standard of fluorescence detection procedures involving a fluorescently labeled binding partner, such as an antibody or other protein with which the MEF2A protein will bind. This could include a substrate upon which the protein acts or an enzyme, which normally acts on the protein. Quantification of isolated protein can be accomplished by other standard methods for isolated protein, such as in situ gel detection, Western blot, or quantitative protein blot. Levels can also be assayed by functional means, such as the effects upon a specific substrate. In the case of the MEF2A protein, this could involve the cleavage of basic amino acids from the C-terminus of the various peptide substrates upon which the MEF2A protein normally acts, or artificial substrates designed for this assay. It is understood that any enzyme activity contained in the MEF2A protein can be used to assess protein levels.

Mutations in MEF2A protein can be analyzed by any of the above or other standard methods known in the art. Protein can be isolated from the cell or analyzed in situ in a cell or tissue sample. Analytic methods include assays for altered electrophoretic mobility, binding properties, tryptic peptide digest, molecular weight, antibody-binding pattern, isoelectric point, amino acid sequence, and any other of the known assay techniques useful for detecting mutations in a protein. Assays include, but are not limited to, those discussed in Varlamov et al., J. Biol. Chem. 271:13981 (1996), incorporated herein by reference for teaching such assays. These include C-terminal arginine binding, acidic pH optima, sensitivity to inhibitors, thermal stability, intracellular distribution, endopeptidase activity, effect on endopeptidase inhibitor, substrate affinity, enzyme kinetics, membrane association, posttranslational modification, active site confirmation, compartmentalization, binding to substrate, secretion, and turnover. Further assays for function can be found in Fricker, J. Cell Biochem. 38:279-289 (1988), and Manser et al., Biochem. J. 267:517-525, (1990), both incorporated by reference for teaching specific functions that can be assayed for mutation in the MEF2A gene.

In vitro techniques for detection of the protein include enzyme linked immunosorbent assays (ELISAs), Western blots, immunoprecipitations and immunofluorescence. Alternatively, the protein can be detected in vivo in a subject by introducing into the subject a labeled anti-MEF2A antibody. For example, the antibody can be labeled with a radioactive marker whose presence and location in a subject can be detected by standard imaging techniques. For detection of specific mutation in the protein, antibodies, or other binding partners, can be used that specifically recognize these alterations. Alternatively, mutations can be detected by direct sequencing of the protein.

Other alterations that can be detected include alterations in post-translational modification. Amino acids, including the terminal amino acids, may be modified by natural processes, such as processing and other post-translational modifications. Common modifications that occur naturally in polypeptides are described in basic texts, detailed monographs, and the research literature, and they are well known to those of skill in the art.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd Ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (Meth. Enzymol. 182: 626-646 (1990)) and Rattan et al. (Ann. N.Y. Acad. Sci. 663:48-62 (1992)).

In addition to detection methods that involve specific physical features, functional characteristics of the MEF2A protein are also useful for detection with known methods. These include changes in biochemistry, such as substrate affinity, enzyme kinetics, membrane association, active site conformation, compartmentalization, forming a complex with substrates or enzymes that act upon the protein, secretion, turnover, pH optima, sensitivity to inhibitors, thermal stability, endopeptidase activity, effects on endopeptidase inhibitors, and any other such functional characteristic that is indicative of a mutation or alteration in post-translational modification. Specific assays can be found in the literature (e.g., see Varlamov et al. (1996) J. Biol. Chem. 271:13981).

MEF2A gene and gene product can be detected in a variety of systems. These include cell-free and cell-based systems in vitro, tissues, such as ex vivo tissues for returning to a patient, in a biopsy, and in vivo, such as in patients being treated, for monitoring clinical trials, and in animal models. Cell-free systems can be derived from cell lines or cell strains in vitro, including recombinant cells, cells derived from patients, subjects involved in clinical trials, and animal models, including transgenic animal models. In one embodiment, MEF2A gene and gene product can also be detected in cell-based systems. This includes cell lines and cell strains in vitro, including recombinant lines and strains containing the MEF2A gene, expanded cells such as primary cultures, particularly those derived from a patient with CAD or MI, subjects undergoing clinical trials, and animal models of CAD or MI including transgenic animals. The MEF2A gene and gene product can also be detected in tissues. These include tissues derived from patients with CAD, subjects undergoing clinical trials, and animal models. In one embodiment, the tissues are those affected in CAD (e.g., myocardial tissue). The MEF2A gene and gene product can also be detected in individual patients with CAD, and subjects undergoing clinical trials, and in animal models of CAD or MI, including transgenic models. Preferred sources of detection include cell and tissue biopsies from individuals affected with CAD or MI or at risk for developing CAD or MI.

In addition to detecting the MEF2A gene or gene products directly, the invention also encompasses the use of compounds that produce a specific effect on a variant MEF2A gene or gene product as a further means of diagnosis. This includes, for example, detection of binding partners, including binding partners specific for variant MEF2A genes or gene products, and compounds that have a detectable effect on a function of MEF2A genes or gene products. For example, an increase in MEF2A levels can be detected by a complex formed between the MEF2A and a binding partner or levels of free MEF2A binding partner. As a further example, abnormally high MEF2A activity could be detected by concurrently high amounts of MEF2A processed substrate.

All these methods of detection can be used in procedures to screen individuals at risk for developing or having CAD or MI. Further, detection of the alterations of the gene or gene products in individuals can serve as a prognostic marker for developing CAD or MI or a diagnostic marker for having CAD or MI when the individuals are not known to have CAD or MI or to be at risk for having CAD or MI. Diagnostic assays can be performed in cell-based systems, and particularly in cells associated with CAD or MI, in intact tissue, such as a biopsy, and nonhuman animals and humans in vivo. Diagnosis can be at the level of nucleic acid or polypeptide.

The invention also encompasses methods for modulating the level or activity of MEF2A gene or gene producta. At the level of the gene, known recombinant techniques can be used to alter the gene in vitro or in situ. Excessive copies of, or all or part of, the MEF2A gene can be deleted. Deletions can be made in any desired region of the gene including transcribed, non-transcribed, coding and non-coding regions. Additional copies of part or all of the gene can also be introduced into a genome. Finally, alterations in nucleotide sequence can be introduced into the gene by recombinant techniques. Alterations include deletions, insertions, inversions, and point mutation. Accordingly, CAD or MI that is caused by a mutated MEF2A gene could be treated by introducing a functional (wild-type) MEF2A gene into the individual. Further, specific alterations could be introduced into the gene and function tested in any given cell type, such as in cell-based models for CAD or MI. Still further, any given mutation can be introduced into a cell and used to form a transgenic animal which can then serve as a model for CAD or MI testing.

Homologously recombinant host cells can also be produced that allow the in situ alteration of endogenous MEF2A polynucleotide sequences in a host cell genome. This technology is more fully described in U.S. Pat. No. 5,641,670, which is herein incorporated by reference. Briefly, specific polynucleotide sequences corresponding to the MEF2A polynucleotides or sequences proximal or distal to a MEF2A gene are allowed to integrate into a host cell genome by homologous recombination where expression of the gene can be affected. In one embodiment, regulatory sequences are introduced that either increase or decrease expression of an endogenous sequence. Accordingly, a MEF2A protein can be produced in a cell not normally producing it, or increased expression of MEF2A protein can result in a cell normally producing the protein at a specific level.

The levels and activity of MEF2A RNA are also subject to modulation. Polynucleotides corresponding to any desired region of the RNA can be used directly to block transcription or translation of MEF2A sequences by means of antisense or ribozyme constructs. Thus, where the disorder is characterized by abnormally high gene expression, these nucleic acids can be used to decrease expression levels. A DNA antisense polynucleotide is designed to be complementary to a region of the gene involved in transcription, preventing transcription and hence production of protein. An antisense RNA or DNA polynucleotide would hybridize to the mRNA and thus block translation of mRNA into protein. An alternative technique involves cleavage by ribozymes containing nucleotide sequences complementary to one or more regions in the mRNA that attenuate the ability of the mRNA to be translated.

The present invention also includes the modulation of nucleic acid expression using compounds that have been discovered by screening the effects of the compounds on MEF2A nucleic acid levels or function.

The invention is further directed to methods for modulating MEF2A protein levels or function. For example, antibodies can be prepared against specific fragments containing sites required for function or against the intact protein. Protein levels can also be modulated by use of compounds discovered in screening techniques in which the protein levels serve as a target for effective compounds. Finally, mutant MEF2A proteins can be functionally affected by the use of compounds discovered in screening techniques that use an alteration of mutant function as an end point.

Modulation can be in a cell-free system. In this context, for example, the assay could involve cleavage of substrate or other indicator of MEF2A activity. Modulation can also occur in cell-based systems. These cells may be permanent cell lines, cell strains, primary cultures, recombinant cells, cells derived from affected individuals, and transgenic animal models of CAD or MI, among others. Modulation can also be in vivo, for example, in patients having the disorder, in subjects undergoing clinical trials, and animal models of CAD or MI, including transgenic animal models. Modulation could be measured by direct assay of the MEF2A gene or gene product or by the results of MEF2A gene and gene product function. All of these methods can be used to affect MEF2A function in individuals having or at risk for having CAD or MI. Thus, the invention encompasses the treatment of CAD or MI by modulating the levels or function of MEF2A genes or gene product.

The invention also encompasses methods for identifying compounds that interact with the MEF2A gene or gene product, particularly to modulate the level or function of the MEF2A gene or gene product. Modulation can be at the level of transcription, translation, or polypeptide function. Accordingly, where levels of MEF2A gene or gene product are abnormally high or low, compounds can be screened for the ability to correct the level of expression. Alternatively, where a mutation affects the function of the MEF2A nucleic acid or protein, compounds can be screened for their ability to compensate for or to correct the dysfunction. In this manner, MEF2A and MEF2A variants can be used to identify agonists and antagonists useful for affecting MEF2A and variant gene expression. These compounds can then be used to affect MEF2A expression or function in individuals with CAD or MI. Thus, these screening methods are useful to identify compounds that can be used for treating CAD or MI.

These compounds are also useful in a diagnostic context in that they can then be used to identify altered levels of MEF2A or MEF2A variants in a cell, tissue, nonhuman animal, and human. For example, compounds specifically interacting with MEF2a nucleic acid or protein to produce a particular result, by producing that result in a cell, tissue, nonhuman animal, or human, indicate that there is a lesion in the MEF2A gene or gene product.

Thus, modulators of gene expression can be identified in a method wherein MEF2A gene or gene product is contacted with a candidate compound and the level or expression of gene or gene product is determined. The level or expression of gene or gene product in the presence of the candidate compound is compared to the level or expression of gene or gene product in the absence of the candidate compound. The candidate compound can then be identified as a modulator of nucleic acid or protein expression based on this comparison and be used, for example, to treat CAD. When the level or expression of gene or gene product is statistically significantly greater in the presence of the candidate compound than in its absence, the candidate compound is identified as a stimulator of levels or expression of the gene or gene product. When levels or product expression are statistically significantly less in the presence of the candidate compound than in its absence, the candidate compound is identified as an inhibitor.

These compounds can be used to test on model systems, including animal models of CAD, and human clinical trial subjects, cells derived from these sources as well as transgenic animal models of CAD. Accordingly, the present invention provides methods of treatment, with the gene or gene product as a target, using a compound identified through drug screening as a modulator to modulate expression of the gene or gene product. Modulation includes both up-regulation (i.e., activation or agonization) or down-regulation (i.e., suppression or antagonization) or nucleic acid expression.

Further, the expression of genes that are up- or down-regulated in response to MEF2A can also be assayed. In this embodiment the regulatory regions of these genes can be operably linked to a reporter gene. Candidate compounds include, for example, 1) peptides such as soluble peptides, including Ig-tailed fusion peptides and members of random peptide libraries (see, e.g., Lam et al., Nature 354:82-84 (1991); Houghten et al., Nature 354:84-86 (1991)) and combinatorial chemistry-derived molecular libraries made of D- and/or L-configuration amino acids; 2) phosphopeptides (e.g., members of random and partially degenerate, directed phosphopeptide libraries, see, e.g., Songyang et al., Cell 72:767-778 (1993)); 3) antibodies (e.g., polyclonal, monoclonal, humanized, anti-idiotypic, chimeric, and single chain antibodies as well as Fab, F(ab′).sub.2, Fab expression library fragments, and epitope-binding fragments of antibodies); and 4) small organic and inorganic molecules (e.g., molecules obtained from combinatorial and natural product libraries).

Any of the biological or biochemical functions mediated by MEF2A can be used in an endpoint assay. These include all of the biochemical or biochemical/biological events described herein, in the references cited herein, incorporated by reference for these endpoint assay targets, and other functions known to those of ordinary skill in the art.

A further aspect of the invention involves pharmacogenomic analysis in the case of polymorphic MEF2A proteins and specific mutants. Pharmacogenomics deal with clinically significant hereditary variations in the response to drugs due to altered drug disposition and abnormal action in affected persons. See, e.g., Eichelbaum, M., Clin. Exp. Pharmacol. Physiol. 23(10-11):983-985 (1996), and Linder, M. W., Clin. Chem. 43(2):254-266 (1997). The clinical outcomes of these variations result in severe toxicity of therapeutic drugs in certain individuals or therapeutic failure of drugs in certain individuals as a result of individual variation in metabolism. Thus, the genotype of the individual can determine the way a therapeutic compound acts on the body or the way the body metabolizes the compound. Further, the activity of drug metabolizing enzymes effects both the intensity and duration of drug action. Thus, the pharmacogenomics of the individual permit the selection of effective compounds and effective dosages of such compounds for prophylactic or therapeutic treatment based on the individual's genotype. Accordingly, in one aspect of the invention, natural variants of the MEF2A protein are used to screen for compounds that are effective against a given allele and are not toxic to the specific patient. Compounds can thus be classed according to their effects against naturally occurring allelic variants. This allows more effective treatment and diagnosis of CAD or MI.

Test systems for identifying compounds include both cell-free and cell-based systems derived from normal and affected tissue, cell lines and strains, primary cultures, animal CAD or MI models, and including transgenic animals. Naturally-occurring cells will express abnormal levels of MEF2A gene or gene product or variants of MEF2A genes or gene products. Alternatively, these cells can provide recombinant hosts for the expression of desired levels of MEF2A gene or gene product or variants of MEF2A gene or gene product. A cell-free system can be used, for example, when assessing the effective agents on nucleic acid or polypeptide function.

For example, in a cell-free system, competition binding assays are designed to discover compounds that interact with the polypeptide. Thus, a compound is exposed to the polypeptide under conditions that allow the compound to bind or to otherwise interact with the polypeptide. Soluble polypeptide is also added to the mixture. If the test compound interacts with the soluble polypeptide, it decreases the amount of complex formed or activity from the target. This type of assay is particularly useful in cases in which compounds are sought that interact with specific regions of the polypeptide. Thus, the soluble polypeptide that competes with the target region is designed to contain peptide sequences corresponding to the region of interest.

To perform cell-free drug screening assays, it is desirable to immobilize either the protein, or fragment, or its target molecule to facilitate separation of complexes from uncomplexed forms of one or both of the proteins, as well as to accommodate automation of the assay. Techniques for immobilizing proteins on matrices can be used in the drug screening assays. In one embodiment, a fusion protein can be provided which adds a domain that allows the protein to be bound to a matrix. For example, glutathione-S-transferase/MEF2A fusion proteins can be adsorbed onto glutathione sepharose beads Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of MEF2A-binding protein found in the bead fraction quantified from the gel using standard electrophoretic techniques. For example, either the polypeptide or its target molecule can be immobilized utilizing conjugation of biotin and streptavidin using techniques well known in the art.

Alternatively, antibodies reactive with the protein but which do not interfere with binding of the protein to its target molecule can be derivatized to the wells of the plate, and the protein trapped in the wells by antibody conjugation. Preparations of an MEF2A-binding protein and a candidate compound are incubated in the MEF2A protein-presenting wells and the amount of complex trapped in the well can be quantitated. Methods for detecting such complexes, in addition to those described above for the GST-immobilized complexes, include immunodetection of complexes using antibodies reactive with the MEF2A protein target molecule, or which are reactive with the MEF2A protein and compete with the target molecule; as well as enzyme-linked assays which rely on detecting an enzymatic activity associated with the target molecule.

Cell-based systems include assay of individual cells or assay of cells in a tissue sample or in vivo. Drug screening assays can be cell-based or cell-free systems. Cell-based systems can be native, i.e., cells that normally express the protein, as a biopsy or expanded in cell culture. In one embodiment, however, cell-based assays involve recombinant host cells expressing the protein. In vivo test systems include, not only individuals involved in clinical trials, but also animal CAD or MI models, including transgenic animals. Single cells include recombinant host cells in which desired altered MEF2A gene or gene products have been introduced. These host cells can express abnormally high or low levels of the MEF2A gene or gene product or mutant versions of the MEF2A gene or gene product. Thus, the recombinant cells can be used as test systems for identifying compounds that have the desired effect on the altered gene or gene product. Mutations can be naturally occurring or constructed for their effect on the course or development of CAD or MI, for example, determined by the model test systems discussed further below. Similarly, naturally-occurring or designed mutations can be introduced into transgenic animals, which then serve as an in vivo test system to identify compounds having a desired effect on MEF2A gene or gene product.

In yet another aspect of the invention, the MEF2A proteins or polypeptides can be used in a “two hybrid” assay (see, for example, U.S. Pat. No. 5,283,317; Zervos et al. (1993) Cell 72:223-232; Madura et al. (1993) Oncogene 8:1693-1696; and Brent WO94/10300), for isolating coding sequences for other cellular proteins which bind to or interact with MEF2A. Briefly, the two hybrid assay relies on reconstituting in vivo a functional transcriptional activator protein from two separate fusion proteins. In particular, the method makes use of chimeric genes which express hybrid proteins. To illustrate, a first hybrid gene comprises the coding sequence for a DNA-binding domain of a transcriptional activator fused in frame to the coding sequence for a MEF2A polypeptide. The second hybrid protein encodes a transcriptional activation domain fused in frame to a sample gene from a cDNA library. If the bait and sample hybrid proteins are able to interact, e.g., form a MEF2A-dependent complex, they bring into close proximity the two domains of the transcriptional activator. This proximity is sufficient to cause transcription of a reporter gene which is operably linked to a transcriptional regulatory site responsive to the transcriptional activator, and expression of the reporter gene can be detected and used to score for the interaction of MEF2A and sample proteins.

Modulators of MEF2A gene or gene product identified according to these assays can be used to treat CAD or MI by treating cells that aberrantly express the gene or gene product. These methods of treatment include the steps of administering the modulators of protein activity in a pharmaceutical composition as described herein, to a subject in need of such treatment. The invention thus provides a method for identifying a compound that can be used to treat autosomal CAD or MI. The method typically includes assaying the ability of the compound to modulate the expression of the MEF2A gene or gene product to identify a compound that can be used to treat the disorder.

The invention is also directed to MEF2A genes or gene products containing alterations that correlate with CAD or MI. These altered genes or gene products can be isolated and purified or can be created in situ, for example, by means of in situ gene replacement techniques In the gene, alterations of this type can be found in any site, transcribed, nontranscribed, coding, and noncoding. Likewise, in the RNA, alterations can be found in both the coding and noncoding regions. In a specific disclosed embodiment, the present invention includes a Δ7aa coding mutation corresponding to a 21-base pair deletion in the MEF2A gene. In another embodiement, the present includes MEF2A proteins that have at least one of a N263S point mutation, a P279L point mutation, and/or a G283D point mutation. The present invention also includes MEF2A gene or gene products that comprises a fragment, preferably a fragment containing the mutation. The invention thus encompasses primers, both wild type and variant, that are useful in the methods described herein. Similarly, ribozymes and antisense nucleic acids can be derived from variants that correlate with CAD or MI or can be derived from the wild type and used in the methods described herein.

The genes and gene products are useful in pharmaceutical compositions for diagnosing or modulating the level or expression of MEF2A gene or gene product in vivo, as in individual patients treated for CAD or MI, subjects in clinical trials, animal CAD or MI models, and transgenic animal CAD or MI models. Thus, these pharmaceutical compositions are useful for testing and treatment. The MEF2A genes or gene products are also useful for otherwise modulating expression of the gene or gene product in cell-free or cell-based systems in vitro. They are further useful in ex vivo applications. The MEF2A genes and gene products are also useful for creating model test systems for CAD or MI, for example, recombinant cells, tissues, and animals. The genes and gene products are also useful in a diagnostic context as comparisons for other naturally-occurring variation in the MEF2A gene or gene product. Accordingly, these reagents can form the basis for a diagnostic kit. Further, specific variants (mutants) are useful for testing compounds that may be effective in the treatment or diagnosis of CAD or MI. Such mutants can also form the basis of a reagent in a test kit, particularly for introduction into a desired cell type or transgenic animal for drug testing. Accordingly, the invention is also directed to isolated and purified polypeptides and polynucleotides.

The present invention thus also relates to compositions based on MEF2A genes or gene products. Compositions also include nucleic acid primers derived from MEF2A mutants, antisense nucleotides derived from these mutants, and ribozymes based on the mutations, and antibodies specific for the mutants. Compositions further include recombinant cells containing any of the mutants, vectors containing the mutants, cells expressing the mutants, fragments of the mutants, and antibodies or other binding partners that specifically recognize the mutation. These compositions can all be combined with a pharmaceutically acceptable carrier to create pharmaceutical compositions useful for detecting or modulating the level or expression of MEF2A gene or gene products and thereby diagnosing or treating CAD or MI.

As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell and still be considered “isolated” or “purified.” The MEF2A polypeptides (or proteins) can be purified to homogeneity. It is understood, however, that preparations in which the polypeptide is not purified to homogeneity are useful and considered to contain an isolated form of the polypeptide. The critical feature is that the preparation allows for the desired function of the polypeptide, even in the presence of considerable amounts of other components. Thus, the invention encompasses various degrees of purity.

In one embodiment, the language “substantially free of cellular material” includes preparations of the protein having less than about 30% (by dry weight) other proteins (i.e., contaminating protein), less than about 20% other proteins, less than about 10% other proteins, or less than about 5% other proteins. When the MEF2A protein is recombinantly produced, it can also be substantially free of culture medium, i.e., culture medium represents less than about 20%, less than about 10%, or less than about 5% of the volume of the protein preparation.

The language “substantially free of chemical precursors or other chemicals” includes preparations of the polypeptide in which it is separated from chemical precursors or other chemicals that are involved in its synthesis. In one embodiment, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

Variants can be naturally-occurring or can be made by recombinant means or chemical synthesis to provide useful and novel characteristics for the polypeptide. This includes preventing immunogenicity from pharmaceutical formulations by preventing protein aggregation. Useful variations further include alteration of binding characteristics. For example, one embodiment involves a variation at the binding site that results in binding but not release, or slower release, of substrate. A further useful variation at the same sites can result in a higher affinity for substrate. Useful variations also include changes that provide for affinity for another substrate. Another useful variation includes one that allows binding but which reduces cleavage of the substrate.

Amino acids that are essential for function of MEF2A transcription factor can be identified by methods known in the art, such as site-directed mutagenesis or alanine-scanning mutagenesis (Cunningham et al., Science 244:1081-1085 (1989)). The latter procedure introduces single alanine mutations at every residue in the molecule. The resulting mutant molecules are then tested for biological activity. Sites that are critical can also be determined by structural analysis such as crystallization, nuclear magnetic resonance or photoaffinity labeling (Smith et al., J. Mol. Biol. 224:899-904 (1992); de Vos et al. Science 255:306-312 (1992)).

The invention also provides antibodies that selectively bind to the MEF2A protein. An antibody is considered to selectively bind, even if it also binds to other proteins that are not substantially homologous with the MEF2A protein. These other proteins share homology with a fragment or domain of the protein. This conservation in specific regions gives rise to antibodies that bind to both proteins by virtue of the homologous sequence. In this case, it would be understood that antibody binding to the MEF2A protein is still selective.

To generate antibodies, an isolated polypeptide is used as an immunogen to generate antibodies using standard techniques for polyclonal and monoclonal antibody preparation. Either the full-length protein or antigenic peptide fragment can be used. Antibodies are preferably prepared from these regions or from discrete fragments in these regions. However, antibodies can be prepared from any region of the peptide as described herein. A preferred fragment produces an antibody that diminishes or completely prevents substrate-binding. Antibodies can be developed against the entire protein or portions of the protein, for example, the substrate binding domain.

Antibodies can be polyclonal or monoclonal. An intact antibody, or a fragment thereof can be used. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

An appropriate immunogenic preparation can be derived from native, recombinantly expressed, protein or chemically synthesized peptides. The antibodies can be used to isolate a MEF2A protein by standard techniques, such as affinity chromatography or immunoprecipitation. The antibodies can facilitate the purification of the natural protein from cells and recombinantly-produced protein expressed in host cells.

The antibodies are useful to detect the presence of protein in cells or tissues to determine the pattern of expression of the protein among various tissues in an organism. The antibodies can be used to detect the protein in situ, in vitro, or in a cell lysate or supernatant in order to evaluate the abundance and pattern of expression. The antibodies can be used to assess abnormal tissue distribution or abnormal expression during development. Antibody detection of circulating fragments of the full length MEF2A protein can be used to identify MEF2A turnover.

Further, the antibodies can be used to assess MEF2A expression in active stages of CAD or in an individual with a predisposition toward CAD. When the disorder is caused by an inappropriate tissue distribution, developmental expression, or level of expression of the MEF2A protein, the antibody can be prepared against the normal MEF2A protein. If a disorder is characterized by a specific mutation in the MEF2A protein, antibodies specific for this mutant protein can be used to assay for the presence of the specific mutant MEF2A protein. However, intracellularly-made antibodies (“intrabodies”) are also encompassed, which would recognize intracellular MEF2A peptide regions.

The antibodies can also be used to assess normal and aberrant subcellular localization of cells in the various tissues in an organism. Antibodies can be developed against the whole MEF2A or portions of the MEF2A. The diagnostic uses can be applied, not only in genetic testing, but also in monitoring a treatment modality. Accordingly, where treatment is ultimately aimed at correcting MEF2A expression level or the presence of aberrant MEF2A and aberrant tissue distribution or developmental expression, antibodies directed against the MEF2A or relevant fragments can be used to monitor therapeutic efficacy. The antibodies are also useful for inhibiting MEF2A function. These uses can also be applied in a therapeutic context. Antibodies can be prepared against specific fragments containing sites required for function or against intact MEF2A associated with a cell.

An “isolated” MEF2A nucleic acid is one that is separated from other nucleic acid present in the natural source of the MEF2A nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. However, there can be some flanking nucleotide sequences, for example up to about 5 KB. The important point is that the nucleic acid is isolated from flanking sequences such that it can be subjected to the specific manipulations described herein such as recombinant expression, preparation of probes and primers, and other uses specific to the nucleic acid sequences.

Moreover, an “isolated” nucleic acid molecule, such as a cDNA molecule, can be substantially free of other cellular material, or culture medium when produced by recombinant techniques, or chemical precursors or other chemicals when chemically synthesized. However, the nucleic acid molecule can be fused to other coding or regulatory sequences and still be considered isolated.

For example, recombinant DNA molecules contained in a vector are considered isolated. Further examples of isolated DNA molecules include recombinant DNA molecules maintained in heterologous host cells or purified (partially or substantially) DNA molecules in solution. Isolated RNA molecules include in vivo or in vitro RNA transcripts of the isolated DNA molecules of the present invention. Isolated nucleic acid molecules according to the present invention further include such molecules produced synthetically.

The MEF2A polynucleotides can encode the mature protein plus additional amino or carboxyl-terminal amino acids, or amino acids interior to the mature polypeptide (when the mature form has more than one polypeptide chain, for instance). Such sequences may play a role in processing of a protein from precursor to a mature form, facilitate protein trafficking, prolong or shorten protein half-life or facilitate manipulation of a protein for assay or production, among other things. As generally is the case in situ, the additional amino acids may be processed away from the mature protein by cellular enzymes.

The MEF2A polynucleotides include, but are not limited to, the sequence encoding the mature polypeptide alone (e.g., SEQ ID NO: 1), the sequence encoding the mature polypeptide and additional coding sequences, such as a leader or secretory sequence (e.g., a pre-pro or pro-protein sequence), the sequence encoding the mature polypeptide, with or without the additional coding sequences, plus additional non-coding sequences, for example introns and non-coding 5′ and 3′ sequences such as transcribed but non-translated sequences that play a role in transcription, mRNA processing (including splicing and polyadenylation signals), ribosome binding and stability of mRNA. In addition, the polynucleotide may be fused to a marker sequence encoding, for example, a peptide that facilitates purification.

Polynucleotides can be in the form of RNA, such as mRNA, or in the form DNA, including cDNA and genomic DNA obtained by cloning or produced by chemical synthetic techniques or by a combination thereof. The nucleic acid, especially DNA, can be double-stranded or single-stranded. Single-stranded nucleic acid can be the coding strand (sense strand) or the non-coding strand (anti-sense strand).

The invention also provides MEF2A nucleic acid molecules encoding the variant polypeptides described herein (e.g., SEQ ID NO: 2). Such polynucleotides may be naturally-occurring, such as allelic variants (same locus), homologs (different locus), and orthologs (different organism), or may be constructed by recombinant DNA methods or by chemical synthesis. Such non-naturally occurring variants may be made by mutagenesis techniques, including those applied to polynucleotides, cells, or organisms. Accordingly, as discussed above, the variants can contain nucleotide substitutions, deletions, inversions and insertions.

Variation can occur in either or both the coding and non-coding regions. The variations can produce both conservative and non-conservative amino acid substitutions. Furthermore, the invention provides polynucleotides that comprise a fragment of the full length MEF2A polynucleotides. The fragment can be single or double stranded and can comprise DNA or RNA. The fragment can be derived from either the coding or the non-coding sequence.

The invention also provides MEF2A nucleic acid fragments that encode epitope bearing regions of the MEF2A proteins described herein. The invention also provides vectors containing the MEF2A polynucleotides. The term “vector” refers to a vehicle, preferably a nucleic acid molecule, that can transport the MEF2A polynucleotides. When the vector is a nucleic acid molecule, the MEF2A polynucleotides are covalently linked to the vector nucleic acid. With this aspect of the invention, the vector includes a plasmid, single or double stranded phage, a single or double stranded RNA or DNA viral vector, or artificial chromosome, such as a BAC, PAC, YAC, OR MAC.

A vector can be maintained in the host cell as an extrachromosomal element where it replicates and produces additional copies of the MEF2A polynucleotides. Alternatively, the vector may integrate into the host cell genome and produce additional copies of the MEF2A polynucleotides when the host cell replicates. The invention provides vectors for the maintenance (cloning vectors) or vectors for expression (expression vectors) of the MEF2A polynucleotides. The vectors can function in prokaryotic or eukaryotic cells or in both (shuttle vectors).

Expression vectors contain cis-acting regulatory regions that are operably linked in the vector to the MEF2A polynucleotides such that transcription of the polynucleotides is allowed in a host cell. The polynucleotides can be introduced into the host cell with a separate polynucleotide capable of affecting transcription. Thus, the second polynucleotide may provide a trans-acting factor interacting with the cis-regulatory control region to allow transcription of the MEF2A polynucleotides from the vector. Alternatively, a trans-acting factor may be supplied by the host cell. Finally, a transacting factor can be produced from the vector itself.

It is understood, however, that in some embodiments, transcription and/or translation of the MEF2A polynucleotides can occur in a cell free system. The regulatory sequence to which the polynucleotides described herein can be operably linked include promoters for directing mRNA transcription. These include, but are not limited to, the left promoter from bacteriophage λ, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

In addition to containing sites for transcription initiation and control, expression vectors can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al., Molecular Cloning: A Laboratory Manual. 2nd. ed., Cold Spring Harbor Laboratory Press, Cold Spring Harbor, N.Y., (1989).

A variety of expression vectors can be used to express a MEF2A polynucleotide. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, poxviruses, pseudorabies viruses, and retroviruses. Vectors may also be derived from combinations of these sources such as those derived from plasmid and bacteriophage genetic elements, e.g. cosmids and phagemids. Appropriate cloning and expression vectors for prokaryotic and eukaryotic hosts are described in Sambrook et al., Molecular Cloning: A Laboratory Manual. 2nd. ed., Cold Spring Harbor Laboratory Press, Cold Spring Harbor, N.Y., (1989).

The regulatory sequence may provide constitutive expression in one or more host cells (i.e. tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

The MEF2A polynucleotides can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art.

The vector containing the appropriate polynucleotide can be introduced into an appropriate host cell for propagation or expression using well-known techniques. Bacterial cells include, but are not limited to, E. coli, Streptomyces, and Salmonella typhimurium. Eukaryotic cells include, but are not limited to, yeast, insect cells such as Drosophila, animal cells such as COS and CHO cells, and plant cells.

As described herein, it may be desirable to express the polypeptide as a fusion protein. Accordingly, the invention provides fusion vectors that allow for the production of the MEF2A polypeptides. Fusion vectors can increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired polypeptide can ultimately be separated from the fusion moiety. Proteolytic enzymes include, but are not limited to, factor Xa, thrombin, and enterokinase. Typical fusion expression vectors include pGEX (Smith et al., Gene 67:31-40 (1988)), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al., Gene 69:301-315 (1988)) and pET 11d (Studier et al., Gene Expression Technology: Methods in Enzymology 185:60-89 (1990)).

Recombinant protein expression can be maximized in a host bacteria by providing a genetic background wherein the host cell has an impaired capacity to proteolytically cleave the recombinant protein. (Gottesman, S., Gene Expression Technology: Methods in Enzymology 185, Academic Press, San Diego, Calif. (1990) 119-128). Alternatively, the sequence of the polynucleotide of interest can be altered to provide preferential codon usage for a specific host cell, for example E. coli. (Wada et al., Nucleic Acids Res. 20:2111-2118 (1992)).

The MEF2A polynucleotides can also be expressed by expression vectors that are operative in yeast. Examples of vectors for expression in yeast e.g., S. cerevisiae include pYepSec1 (Baldari, et al., EMBO J. 6:229-234 (1987)), pMFa (Kurjan et al., Cell 30:933-943(1982)), pJRY88 (Schultz et al., Gene 54:113-123 (1987)), and pYES2 (Invitrogen Corporation, San Diego, Calif.).

The MEF2A polynucleotides can also be expressed in insect cells using, for example, baculovirus expression vectors. Baculovirus vectors available for expression of proteins in cultured insect cells (e.g., Sf 9 cells) include the pAc series (Smith et al., Mol. Cell Biol. 3:2156-2165 (1983)) and the pVL series (Lucklow et al., Virology 170:31-39 (1989)).

In certain embodiments of the invention, the polynucleotides described herein are expressed in mammalian cells using mammalian expression vectors. Examples of mammalian expression vectors include pCDM8 (Seed, B. Nature 329:840(1987)) and pMT2PC (Kaufman et al, EMBO J. 6:187-195 (1987)).

The expression vectors listed herein are provided by way of example only of the well-known vectors available to those of ordinary skill in the art that would be useful to express the MEF2A polynucleotides. The person of ordinary skill in the art would be aware of other vectors suitable for maintenance propagation or expression of the polynucleotides described herein. These are found for example in Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989.

The invention also encompasses vectors in which the nucleic acid sequences described herein are cloned into the vector in reverse orientation, but operably linked to a regulatory sequence that permits transcription of antisense RNA. Thus, an antisense transcript can be produced to all, or to a portion, of the polynucleotide sequences described herein, including both coding and noncoding regions. Expression of this antisense RNA is subject to each of the parameters described above in relation to expression of the sense RNA (regulatory sequences, constitutive or inducible expression, tissue-specific expression).

The invention also relates to recombinant host cells containing the vectors described herein. Host cells therefore include prokaryotic cells, lower eukaryotic cells such as yeast, other eukaryotic cells such as insect cells, and higher eukaryotic cells such as mammalian cells.

The recombinant host cells are prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989).

Host cells can contain more than one vector. Thus, different nucleotide sequences can be introduced on different vectors of the same cell. Similarly, the MEF2A polynucleotides can be introduced either alone or with other polynucleotides that are not related to the MEF2A polynucleotides such as those providing trans-acting factors for expression vectors. When more than one vector is introduced into a cell, the vectors can be introduced independently, co-introduced or joined to the MEF2A polynucleotide vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Vectors generally include selectable markers that enable the selection of the subpopulation of cells that contain the recombinant vector constructs. The marker can be contained in the same vector that contains the polynucleotides described herein or may be on a separate vector. Markers include tetracycline or ampicillin-resistance genes for prokaryotic host cells and dihydrofolate reductase or neomycin resistance for eukaryotic host cells. However, any marker that provides selection for a phenotypic trait will be effective. While the mature proteins can be produced in bacteria, yeast, mammalian cells, and other cells under the control of the appropriate regulatory sequences, cell free transcription and translation systems can also be used to produce these proteins using RNA derived from the DNA constructs described herein.

Where secretion of the polypeptide is desired, appropriate secretion signals are incorporated into the vector. The signal sequence can be endogenous to the MEF2A proteins or heterologous to these proteins. Where the protein is not secreted into the medium, the protein can be isolated from the host cell by standard disruption procedures, including freeze thaw, sonication, mechanical disruption, use of lysing agents and the like. The polypeptide can then be recovered and purified by well-known purification methods including ammonium sulfate precipitation, acid extraction, anion or cationic exchange chromatography, phosphocellulose chromatography, hydrophobic-interaction chromatography, affinity chromatography, hydroxylapatite chromatography, lectin chromatography, or high performance liquid chromatography.

It is also understood that depending upon the host cell in recombinant production of the polypeptides described herein, the polypeptides can have various glycosylation patterns, depending upon the cell, or may be non-glycosylated as when produced in bacteria. In addition, the polypeptides may include an initial modified methionine in some cases as a result of a host-mediated process.

The host cells expressing the polypeptides described herein, and particularly recombinant host cells, have a variety of uses. First, the cells are useful for producing MEF2A proteins or polypeptides that can be further purified to produce desired amounts of MEF2A protein or fragments. Thus, host cells containing expression vectors are useful for polypeptide production. Host cells are also useful for conducting cell based assays involving the MEF2A or MEF2A fragments. Thus, a recombinant host cell expressing a native MEF2A is useful to assay for compounds that stimulate or inhibit MEF2A function.

Host cells are also useful for identifying MEF2A mutants in which these functions are affected. If the mutants naturally occur, host cells containing the mutations are useful to assay compounds that have a desired effect on the mutant MEF2A (for example, stimulating or inhibiting function) which may not be indicated by their effect on the native MEF2A.

Recombinant host cells are also useful for expressing the chimeric polypeptides described herein to assess compounds that activate or suppress activation by means of a heterologous amino terminal extracellular domain (or other binding region). Alternatively, a heterologous region spanning the entire transmembrane domain (or parts thereof) can be used to assess the effect of a desired amino terminal extracellular domain (or other binding region) on any given host cell. In this embodiment, a region spanning the entire transmembrane domain (or parts thereof) compatible with the specific host cell is used to make the chimeric vector. Alternatively, a heterologous carboxy terminal intracellular, e.g., signal transduction, domain can be introduced into the host cell.

Further, mutant MEF2As can be designed in which one or more of the various functions is engineered to be increased or decreased used to augment or replace MEF2A proteins in an individual. Thus, host cells can provide a therapeutic benefit by replacing an aberrant MEF2A or providing an aberrant MEF2A that provides a therapeutic result. In one embodiment, the cells provide MEF2A that is abnormally active.

Homologously recombinant host cells can also be produced that allow the in situ alteration of endogenous MEF2A polynucleotide sequences in a host cell genome. This technology is more fully described in U.S. Pat. No. 5,641,670. Briefly, specific polynucleotide sequences corresponding to the MEF2A polynucleotides or sequences proximal or distal to a MEF2A gene are allowed to integrate into a host cell genome by homologous recombination where expression of the gene can be affected. In one embodiment, regulatory sequences are introduced that either increase or decrease expression of an endogenous sequence. Accordingly, a MEF2A protein can be produced in a cell not normally producing it, or increased expression of MEF2A protein can result in a cell normally producing the protein at a specific level.

In one embodiment, the host cell can be a fertilized oocyte or embryonic stem cell that can be used to produce a transgenic animal containing the altered MEF2A gene. Alternatively, the host cell can be a stem cell or other early tissue precursor that gives rise to a specific subset of cells and can be used to produce transgenic tissues in an animal. See also Thomas et al., Cell 51:503 (1987) for a description of homologous recombination vectors. The vector is introduced into an embryonic stem cell line (e.g., by electroporation) and cells in which the introduced gene has homologously recombined with the endogenous MEF2A gene is selected (see e.g., Li, E. et al., Cell 69:915 (1992)). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos. WO 90/11354; WO 91/01140; and WO 93/04169.

The genetically engineered host cells can be used to produce non-human transgenic animals. A transgenic animal is preferably a mammal, for example a rodent, such as a rat or mouse, in which one or more of the cells of the animal include a transgene. A transgene is exogenous DNA which is integrated into the genome of a cell from which a transgenic animal develops and which remains in the genome of the mature animal in one or more cell types or tissues of the transgenic animal. These animals are useful for studying the function of a MEF2A protein and identifying and evaluating modulators of MEF2A protein activity. Other examples of transgenic animals include non-human primates, sheep, dogs, cows, goats, chickens, and amphibians.

In one embodiment, a host cell is a fertilized oocyte or an embryonic stem cell into which MEF2A polynucleotide sequences have been introduced. A transgenic animal can be produced by introducing nucleic acid into the male pronuclei of a fertilized oocyte, e.g., by microinjection, retroviral infection, and allowing the oocyte to develop in a pseudopregnant female foster animal. Any of the MEF2A nucleotide sequences described herein, especially the altered sequences, can be introduced as a transgene into the genome of a non-human animal, such as a mouse.

Any of the regulatory or other sequences useful in expression vectors can form part of the transgenic sequence. This includes intronic sequences and polyadenylation signals, if not already included. A tissue-specific regulatory sequence(s) can be operably linked to the transgene to direct expression of the MEF2A protein to particular cells.

Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals caring a transgene can further be bred to other transgenic animals carrying other transgenes. A transgenic animal also includes animals in which the entire animal or tissues in the animal have been produced using the homologously recombinant host cells described herein.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g. Lakso et al. PNAS 89:6232-6236 (1992). Another example of a recombinase system is the FLP recombinase system of S. cerevisiae (O'Gorman et al. Science 251:1351-1355 (1991). If a cre/loxP recombinase system is used to regulate expression of the transgene, animals containing transgenes encoding both the Cre recombinase and a selected protein is required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. Nature 385:810-813 (1997) and PCT International Publication Nos. WO 97/07668 and WO 97/07669. In brief, a cell, e.g., a somatic cell, from the transgenic animal can be isolated and induced to exit the growth cycle and enter GO phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to a pseudopregnant female foster animal. The offspring born of this female foster animal will be a clone of the animal from which the cell, e.g., the somatic cell, is isolated.

Transgenic animals containing recombinant cells that express the polypeptides described herein are useful to conduct the assays described herein in an in vivo context. Accordingly, the various physiological factors that are present in vivo and that could effect ligand binding, MEF2A activation, and signal transduction, may not be evident from in vitro cell free or cell based assays. Accordingly, it is useful to provide non-human transgenic animals to assay in vivo MEF2A function, the effect of specific mutant MEF2As on MEF2A function, and the effect of chimeric MEF2As. It is also possible to assess the effect of null mutations, that is mutations that substantially or completely eliminate one or more MEF2A functions.

The MEF2A nucleic acid molecules, protein (particularly fragments, such as the domains that interact with other cellular components), modulators of the nucleic acid and protein, and especially binding partners, and antibodies (also referred to herein as “active compounds”) can be incorporated into pharmaceutical compositions suitable for administration to a subject, e.g., a human. Such compositions typically comprise the nucleic acid molecule, protein, modulator, or antibody and a pharmaceutically acceptable carrier.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be compatible with its intended route of administration. Examples of routes of administration include parenteral, (e.g., intravenous, intradermal, subcutaneous), oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a MEF2A protein or anti-MEF2A antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant (e.g., a gas such as carbon dioxide) or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one aspect of the invention, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see e.g., Chen et al., PNAS 91:3054-3057 (1994)). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells (e.g., retroviral vectors) the pharmaceutical preparation can include one or more cells which produce the gene delivery system.

The pharmaceutical compositions can be included in a container, pack, or dispenser together with instructions for administration. This invention may be embodied in many different forms and should not be construed as limited to the embodiments set forth herein; rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated.

The following examples are included to demonstrate various aspects of the invention. Those of skill in the art should, in light of the present disclosure, appreciate that many changes can be made in the specific aspects which are disclosed and still obtain a like or similar result without departing from the spirit and scope of the invention.

### EXAMPLES

### Example 1

We studied a large family with 13 patients that demonstrate an autosomal dominant pattern of CAD/MI (kindred QW1576 in FIG. 1, Table 1). CAD was defined as any previous or current evidence of MI (based on the existence of at least two of the following: chest pain of >30 minutes duration, ECG patterns consistent with acute myocardial infarction, or significant elevation of cardiac enzymes), percutaneous coronary angioplasty (PTCA), coronary artery bypass surgery (CABG), or coronary angiography with >70% stenosis. PTCA is one of the most common non-surgical treatment for opening obstructed coronary arteries. A catheter with a deflated balloon at its tip is inserted and advanced into the narrowed part of a coronary artery. The balloon is then inflated, which compresses the plaque and enlarge the inner diameter of the coronary artery to allow blood to flow more easily. A stent is sometimes placed to keep the arteries open. The balloon is then deflated and the catheter removed. Five of the patients in kindred QW1576 had PTCA. CABG is the most commonly performed open heart operation to bypass blockages or obstructions of the coronary arteries. Four of the patients in the family underwent CABG. Four affected members had premature CAD/MI at under 45 years of age: individual I.1, MI at age of 45 years; III.1, PTCA at 35; III.4, MI and CABG at 42; III.6, MI at 40 (Table 1). No hypercholesterolemia was present in any patient. We carried out a genome-wide linkage scan with 382 ABI LMS-MD10 microsatellite polymorphic markers spanning chromosomes 1-22 with an average interval of 10 cM. The only positive linkage was identified for marker D15S120 with a Lod score of 4.19 at a recombination fraction of 0. Further haplotype analysis with markers D15S1014, D15S212, and D15S87 verified the observed linkage (FIG. 1). These data identify a significant linkage to autosomal dominant CAD/MI on chromosome 15q26 (this locus is designated as adCAD/MI1 for the first autosomal dominant CAD and MI locus).

The candidate adCAD/MI1 region contains approximately 93 genes (table S2), which consists of 43 known genes and 50 hypothetical genes. Among the known genes, MEF2A, which encodes a member of the myocyte enhancer factor-2 (MEF2) family of transcription factors, became a strong candidate as MEF2A mRNA was detected in blood vessels around the neural tube during mouse early embryogenesis and MEF2A protein was proposed as an early embryonic marker for cells of the vasculature. In vertebrates, there are four MEF2 factors, including MEF2A, MEF2B, MEF2C, and MEF2D ({Black, 1998 1103/id;McKinsey, 2002 1104/id}). They belong to the MADS-box family of transcriptional regulators with similar functional domain structures. At the N-termini, MEF2 factors contain a 57-amino acid MADS domain that mediates dimerization and DNA binding to AT-rich sequences [CTA(A/T)4TAG/A]. Adjacent is a 29 amino acid MEF2-specific domain that is required for high-affinity DNA binding, dimerization and cofactor interactions. The C-termini are required for transcription activation and nuclear localization. MEF2 proteins interact with a variety of other transcription factors to regulate the expression of the downstream target genes. These factors include MyoD, GATA, NFAT, 14-3-3, ERK, and p300/PCAF that stimulates MEF2 activity, and HDAC4, 5, 7, 9, MITR, and Cabin that suppress MEF2 function. MEF2 genes are expressed and are functional as early as days 7.5 to 9 postcoitum (d.p.c) during early mouse embryogenesis. After birth, MEF2A, MEF2B, and MEF2D genes are expressed ubiquitously, whereas MEF2C expression is limited to skeletal muscle, brain, and spleen. MEF2 genes are involved in linking calcium-dependent signaling pathways to the genes involved in cell division, differentiation, and death.

The MEF2A gene became a strong candidate for CAD/MI based on its chromosome 15q26 location (FIG. 1) and emerging evidence indicating that MEF2A protein can serve as an early marker for cells of the vasculature. We, therefore, undertook a systematic mutational screening in the entire MEF2A gene. The MEF2A gene contains 11 exons. All of the exons of the MEF2A gene (including exon-intron boundaries) were amplified by PCR using intronic primers (Table S1) and directly sequenced. A novel 21-bp deletion was identified in exon 11 in all ten living affected members in the family (FIG. 2). The 21-bp deletion results in a deletion of 7 amino acids of MEF2A (ΔQ₄₄₀P₄₄₁P₄₄₂Q₄₄₃P₄₄₄Q₄₄₅P₄₄₆ or Δ7aa). These 7 amino acids are highly conserved among MEF2As from the humans, the mouse (QPPQPQP), the pig (pqPQPQa), and Ateles belzebuth chamek (QPqQPQP). The Δ7aa is located in the conserved C-terminus region between MEF2A and MEF2C, which has been demonstrated to be important for nuclear localization of these two proteins. The 21-bp deletion was not identified in the family members with a normal phenotype. Furthermore, the 21-bp deletion was absent in 119 control individuals. The control individuals were selected among >6,000 individuals examined at our Catheterization Laboratories. Only those who were >55 years old and whose coronary angiography showed no luminal stenosis were chosen as controls. These genetic data strongly suggest that the 21-bp deletion of MEF2A causes CAD and MI in a large family.

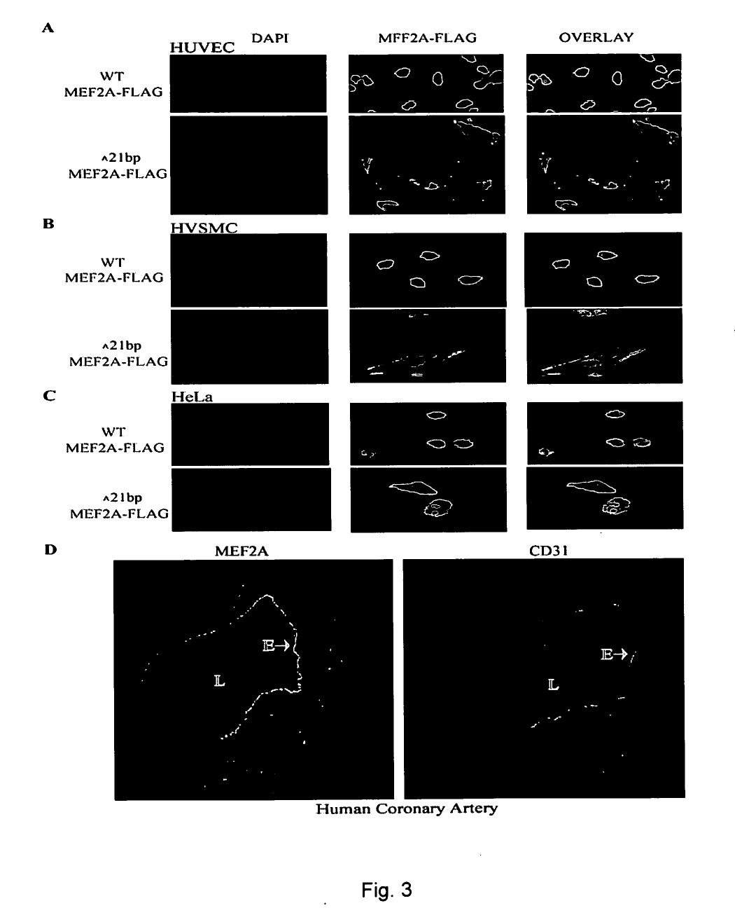

The functional consequences of the 7-amino acid deletion (Δ7aa) of MEF2A were explored. We hypothesized that Δ7aa may cause a conformational change of the MEF2A protein and result in protein trafficking defects. Such a defect will prevent MEF2A from exerting its function as a transcription factor. To test this hypothesis, we expressed wild type and mutant MEF2A proteins tagged with a FLAG-epitope into human umbilical vascular endothelial cells (HUVEC), smooth muscle and HeLa cells, and studied cellular localization of MEF2A by immunofluorescence staining with a monoclonal anti-FLAG antibody. As expected, wild type MEF2A is localized completely into the nucleus (FIG. 3A-C, WT, green signal). However, the 21-bp deletion causes a defect in MEF2A trafficking with block of MEF2A entry into the nucleus in all three cell types examined (FIG. 3A-C). These results demonstrate that the 21-bp deletion is a functional mutation that disrupts the nuclear localization of MEF2A, which would alter the expression profile of MEF2A-target genes.

The mechanism of the retention of the deletion mutant MEF2A in the cytoplasm is not clear. This deleted region may play a critical role in nuclear localization of MEF2A. It is interesting to note that the corresponding region of MEF2C has also been found to play an important role in its nuclear localization or nuclear retention. Alternatively, the 7-amino acid deletion may result in a misfolded protein that impairs MEF2A transport and trafficking. Some partially folded or incorrectly folded mutant MEF2A may generate aggregates of varying size, which may have a difficulty entering the nucleus.

The functional consequence of the 7-amino acid deletion (Δ7aa) of MEF2A was also explored by transcription activation assay. It has been demonstrated that the ANF₋₇₀₀ promoter can be activated by the cooperation between MEF2A and GATA-1, a member of the GATA family of zinc-finger transcription factors. Thus, we used the ANF₋₇₀₀ promoter as a reporter gene (the region from −700 bp to +1 bp from the transcriptional start site of ANF was fused to the luciferase gene) to analyze the effect of Δ7aa on MEF2A transcription activation. The ANF₋₇₀₀ reporter gene was co-transfected with the wild type or mutant MEF2A expression construct alone or in combination into HeLa cells. Transcription activity was examined and expressed as relative luciferase units. As shown in FIG. 4, MEF2A with Δ7aa has only ⅓ of wild type MEF2A transcription activity, indicating that the 7 amino acid deletion identified in kindred QW1576 is a functional mutation that reduces transcription activation by MEF2A. Either wild type MEF2A or GATA-1 alone activated expression of the ANF₇₀₀ promoter, but co-transfection of MEF2A or GATA-1 showed synergistic activation of the ANF₋₇₀₀ promoter as reported previously. However, synergistic activation by MEF2A and GATA-1 was abolished by Δ7aa in MEF2A (FIG. 4), further indicating that Δ7aa is a functional mutation. Co-expression of mutant MEF2A with wild type MEF2A showed the similar transcription activity to mutant MEF2A alone (FIG. 4). The synergistic activation of transcription by MEF2A and GATA-1 was also abolished by co-expression of the mutant Δ7aa MEF2A with normal wild type MEF2A (FIG. 4). Together, these data suggest that Δ7aa acts by a dominant-negative mechanism (the mutant form of MEF2A interferes with the function of the normal wild type MEF2A or GATA-1 through a ‘poison pill’-type mechanism). The dominant-negative effect of the mutant Δ7aa MEF2A can be explained by the findings that MEF2A acts as a dimer or as a complex with GATA factors.

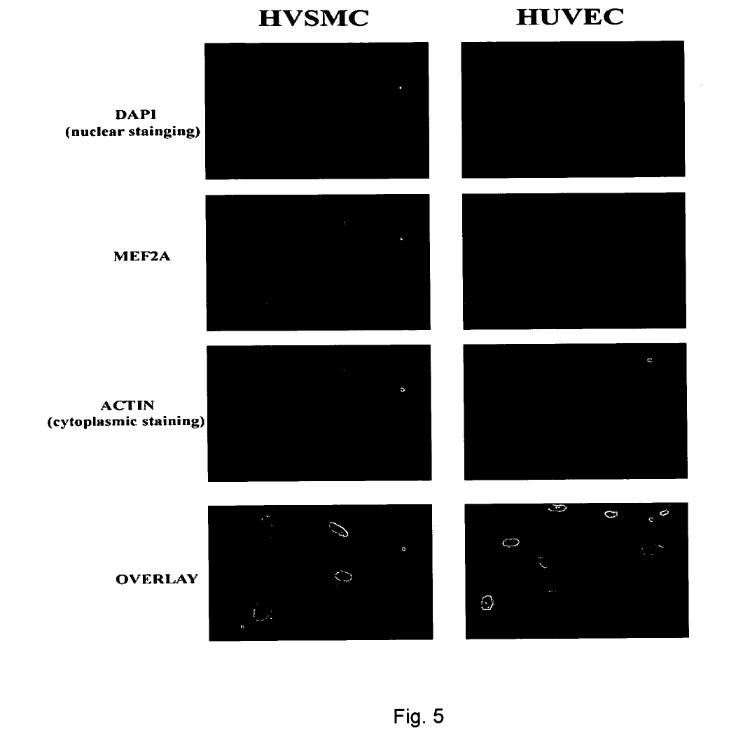

We next tested whether MEF2A is expressed in human coronary arteries, the target organ of coronary artery disease and myocardial infarction. Immunostaining using the anti-MEF2A polyclonal antibody detected very strong MEF2A protein expression at the endothelial cell layer of coronary arteries (FIG. 3D). This pattern of expression is similar to that observed with a monoclonal antibody for CD31 (PECAM), an endothelial cell marker. Immunostaining and reverse-transcription PCR also detected MEF2A expression in human umbilical vascular endothelial cells (FIG. 5). Consistent with these results, in situ immunohistochemistry of whole mount mouse embryos at different stages of development using a polyclonal antibody specific to MEF2A revealed that MEF2A protein is expressed as early as day 8.5 postcoitum in cells of the embryonic vasculature (serving as an early marker for cells of vasculature), and in the aorta, inter-somitic arteries, vessels of the head and capillary plexus in the dorsal region of old embryos. Embryonic regions with MEF2A protein expression were also immunostained with an antibody specific for the Von Willebrand factor (vWF, an endothelial cell marker). The overall expression pattern of MEF2A is similar to that of vascular endothelial growth factor receptor 2 in endothelial cell precursors. These studies suggest that MEF2A can be an early marker for vasculogenesis and may play an important role in controlling vascular morphogenesis.

Collectively, the above data implicate an important biological role of the MEF2A transcription factor in endothelial cell development and function. The pathogenesis of coronary artery disease and myocardial infarction is associated sequentially with endothelial dysfunction and rupture, which promotes the diapedesis of monocytes and exposes the subendothelial matrix to thrombosis, respectively. The transmigration of monocytes, and their differentiation into foam cells, has been known to be a critical path in the genesis of atherosclerotic plaque. A genetic defect in MEF2A may lead to a defective or abnormal vascular endothelium, which could trigger the initiation of atherogenesis.

In addition to expression in endothelial cells, MEF2A mRNA was also detected in cultured proliferating rat smooth muscle cells (SMCs). We have also detected expression of MEF2A protein in the nuclei of proliferating SMCs (FIG. 5). In the rat model of arterial injury modeling clinical restenosis, in situ hybridization with carotid arteries showed that strong expression of MEF2A was detected in the neointimal cells close to the lumen (cells arising as a consequence of deendothelialization). MEF2A signal from the medial cells is at or near the background level. Immunohistochemistry showed that MEF2A protein expression was restricted to the neointima cells close the lumen and medial SMCs do not express detectable MEF2A protein. These studies suggest that MEF2A is expressed in proliferating SMCs, but not in differentiated SMCs in the medial layer of vessels. The increased SMC proliferation was found to be associated with accelerated atherosclerosis. Therefore, the 7aa deletion in MEF2A may affect the activity of proliferating SMCs, influencing the progress of artherogenesis.

Mice deficient in MEF2A have been created. Homozygous MEF2a^{-/-} mice in the 129sv genetic background die suddenly within the first week of life, however, mice on a mixed genetic background survive. Dilation of the right ventricle was detected for the homozygous MEF2a^{-/-} mice with sudden death at necropsy, but not before death. Mice that escaped the perinatal sudden death and reached adulthood are also susceptible to sudden death. These mice had decreased level of mitochondria without any structural heart abnormalities. The cause of the sudden death phenotype in homozygous MEF2a^{-/-} mice remains largely unknown. Further phenotypic characterization of MEF2a^{-/-} mice will determine whether these mice show any phenotype related to human CAD and MI. Heterozygous MEF2a^{+/-} mice exhibit a normal phenotype. The phenotypic difference between heterozygous MEF2a^{+/-} mice and the human patients with heterozygous MEF2A 21-bp deletion may reflect the inherent differences between the two species and/or specific effects of the 7-amino acid deletion in MEF2A.

Similar to other cardiovascular diseases such as long QT syndrome and hypertrophic cardiomyopathy, familial CAD/MI is likely to be genetically heterogeneous. Our linkage analysis suggests that three other large families with CAD and MI are not linked to the chromosome 15q26 adCAD/MI1 locus. Single-strand conformation polymorphism (SSCP) analysis failed to detect mutations in MEF2A in >50 sporadic patients with CAD and MI. MEF2A mutations may, therefore, be a rare cause of CAD and MI, however, the true prevalence rate of MEF2A mutations in the CAD/MI patient population will be revealed by future studies with a large sample size. Finally, although unlikely, we cannot exclude the possibility that another mutation in a yet unidentified gene, which is in disequilibrium with the MEF2A 7aa deletion, also contributes to the development of CAD/MI.

In summary, our results define a novel genetic pathway and provide a molecular mechanism for the pathogenesis of familial CAD and MI. Our findings open new avenues for understanding the complex pathogenic mechanisms of CAD and MI. The implications are that the hitherto unsuspected gene MEF2A, or related genes in the MEF2A signaling pathway, may underlie other forms of atherosclerotic disease, and furthermore, that genes regulating endothelial development and function may be pathophysiologically relevant to this complex disease process.

### REFERENCES AND NOTES

1. C. J. Murray and A. D. Lopez, Lancet 349, 1498-1504 (1997).
2. American Heart Association, “Heart disease and stroke statistics-2003 update” (American Heart Association, 2002).
3. A. J. Lusis, Nature 407, 233-241 (2000).
4. J. J. Nora, R. H. Lortscher, R. D. Spangler, A. H. Nora, W. J. Kimberling, Circulation 61, 503-508 (1980).
5. J. M. Schildkraut, R. H. Myers, L. A. Cupples, D. K. Kiely, W. B. Kannel, Am. J. Cardiol. 64, 555-559 (1989).
6. Wang Q and Pyeritz R E, in Textbook of Cardiovascular Medicine, Topol E J, Ed. (Lippincott Williams & Wilkins, New York, N.Y., 2000), vol. 3.
7. U. Broeckel et al., Nat. Genet. 30, 210-214 (2002).
8. S. Francke et al., Hum. Mol. Genet. 10, 2751-2765 (2001).
9. S. B. Harrap et al., Arterioscler. Thromb. Vasc. Biol. 22, 874-878 (2002).
10. P. Pajukanta et al., Am. J. Hum. Genet. 67, 1481-1493 (2000).
11. B. L. Black, J. D. Molkentin, E. N. Olson, Mol. Cell Biol. 18, 69-77 (1998).
12. T. A. McKinsey, C. L. Zhang, E. N. Olson, Trends Biochem. Sci. 27, 40-47 (2002).
13. D. G. Edmondson, G. E. Lyons, J. F. Martin, E. N. Olson, Development 120, 1251-1263 (1994).
14. R. E. Breitbart et al., Development 118, 1095-1106 (1993).
15. Y. T. Yu et al., Genes Dev. 6, 1783-1798 (1992).
16. S. V. Subramanian and B. Nadal-Ginard, Mech. Dev. 57, 103-112 (1996).
17. S. Borghi et al., J. Cell Sci. 114, 4477-4483 (2001).
18. M. Bucciantini et al., Nature 416, 507-511 (2002).

19. F. J. Naya et al., Nat. Med. 8, 1303-1309 (2002).

TABLE 1
Clinical characteristics of the family members
in a family with CAD and MI.

| Individual | Age | |
|---|---|---|
| ID# | (years) | Clinical Diagnosis (age of diagnosis in years) |
| I.1 | — | MI (45) |
| II.1 | 63 | MI (63), PTCA (63) |
| II.2 | — | MI (65), CABG (65) |
| II.4 | 81 | MI (80), CABG (65), CATH ((>70% |
| | | stenosis; 65) |
| II.5 | 81 | Normal |
| II.6 | 61 | MI (61), CABG (61) |
| II.8 | 77 | MI (61), CATH (>70% stenosis; 61) |
| II.10 | 72 | Normal |
| II.11 | 68 | PTCA (68) |
| II.12 | 63 | MI (63) |
| II.13 | 65 | PTCA (64) |
| III.1 | 51 | PTCA (35) |
| III.2 | 49 | Uncertain (no symptoms, female, but ≦55 years |
| | | of age) |
| III.3 | 47 | PTCA (46) |
| III.4 | 49 | MI (42), CABG (42) |
| III.5 | 50 | Uncertain (no symptoms, female, but ≦55 years |
| | | of age) |
| III.6 | — | MI (40) |
| III.7 | 54 | Normal (no symptoms, male, ≧50 years of age) |
| III.8 | 50 | Normal (no symptoms, male, ≧50 years of age) |
| III.9 | 50 | Normal (no symptoms, male, ≧50 years of age) |
| III.10 | 46 | Uncertain (no symptoms, female, but ≦55 years |
| | | of age) |

MI, myocardial infarction; PTCA, percutaneous coronary angioplasty; CABG, coronary artery bypass surgery.
Materials and Methods
Study Subjects and Isolation of Genomic DNA

The study participants were identified at the Department of Cardiovascular Medicine at the Cleveland Clinic Foundation. CAD was defined as any previous or current evidence of significant atherosclerotic coronary artery disease (defined as myocardial infarction (MI), percutaneous coronary angioplasty (PTCA), coronary artery bypass surgery (CABG) or coronary angiography with >70% stenosis) (1). Diagnosis of MI was based on the existence of at least two of the following: chest pain of ≧30 minutes duration, ECG patterns consistent with acute MI, or significant elevation of cardiac enzymes (1). Exclusion criteria include hypercholesterolemia, insulin-dependent diabetes, childhood hypertension, substance abuse, and congenital heart disease. Informed consent was obtained from all participants or their guardians, in accordance with standards established by the Cleveland Clinic Foundation Institutional Review Board on Human Subjects.

The normal controls are defined as individuals at the age of ≧55 years whose coronary angiography showed no luminal stenosis. These controls were selected among >6,000 individuals in the GeneBank at the Cleveland Clinic Heart Center, which is a registry of data in conjunction with a repository of DNA/Serum/and plasma for the individuals undergoing coronary catheterization.

Genomic DNA was prepared from the whole blood with the DNA Isolation Kit for Mammalian Blood (Roche Diagnostic Co., Indianapolis, Ind.).

Genotyping

The genome-wide linkage scan includes 382 polymorphic microsatellite markers on chromosomes 1-22 (ABI PRISM Linkage Mapping Set-MD10). Additional markers were identified at the Genethon database. Markers were genotyped using an ABI 3100 Genetic Analyzer (Applied Biosystems, Foster City, Calif.), and genotypes were analyzed using GeneMapper 2 Software (Applied Biosystems, Foster City, Calif.). Pairwise logarithm of the odds (LOD) scores were calculated with the Linkage Package 5.2 assuming the autosomal dominanat inheritance (2-4).

Mutation Analysis

The genomic structure of the MEF2A gene was determined by comparing its cDNA sequence to the genomic sequence using BLAST. PCR primers were designed based on the flanking intronic sequences of each exon. The complete coding region and the intron splice sites of the MEF2A gene were amplified by PCR. Amplified products were purified using the QIAquick PCR Purification Kit (QIAGEN Inc., Valencia, Calif.) and sequenced with forward and reverse primers by an ABI3100 Genetic Analyzer (Applied Biosystems, Foster City, Calif.).

Single-strand conformational polymorphism (SSCP) analysis was used to confirm the identified mutation and to test the presence/absence of the mutation in the normal controls as described previously (3-6).

Plasmid Constructs and Mutagenesis

The full-length MEF2A cDNA was cloned into the expression vector pcDNA3. The MEF2A expression construct has the FLAG-epitope tagged at the C-terminus (kindly provided by Dr. Eric N. Olson at University of Texas Southwestern Medical Center). The full-length GATA-1 cDNA was isolated by RT-PCR, and cloned into pcDNA3, resulting in the GATA-1 expression construct.

The 21-bp deletion of MEF2A (ΔQ₄₄₀P₄₄₁P₄₄₂Q₄₄₃P₄₄₄Q₄₄₅P₄₄₆) was introduced into the wild type construct by PCR-based site-directed mutagenesis (7) and verified by DNA sequencing.

The region from −700 bp to +1 bp upstream from the transcription start site of the human atrial natriuretic factor (ANF) promoter was PCR-amplified and cloned into the pGL3-Basic vector, resulting in the ANF₋₇₀₀-Luc reporter gene.

Immunofluorescence Staining

Human umbilical vascular endothelial cells (HUVECs), vascular smooth muscle cells (HVSMCs) and HeLa cells were grown to 90% confluence in Dulbecco's minimum essential medium (DMEM) supplemented with 10% fetal bovine serum, and transfected with Lipofectamine 2000 (Invitrogen) and 500 ng of DNA (8-9). Transfected cells were seeded on Lab-Tek II chamber slides (Nalge Nunc International, Naperville, Ill.) at a density of 1×10⁵ cells and incubated at 37° C. and 5% CO₂ for 24 hours. Cells were then fixed in 4% paraformadehyde, washed with PBS, and incubated with the primary antibody (1:2000 dilution) in PBS/5% nonfat milk at 4° C. overnight. The mouse anti-FLAG M2 primary antibody (Sigma, St. Louis, Mo.) recognizes the FLAG-tagged MEF2A protein. The secondary antibody, a FITC-conjugated sheep anti-mouse IgG (1:2000 dilution) was then added and incubated at room temperature for 1 hour.

Tissue section immunostaining for coronary arteries was carried out as previously described (10) with minor modifications. Briefly, frozen human coronary artery sections were fixed with 4% paraformaldehyde, treated with 0.5% Triton X-100 PBS, blocked in blocking buffer (PBS/5% nonfat milk), and then incubated with the primary antibody (1:250 dilution) at 4° C. overnight. The sections were then incubated with the FITC-conjugated anti-rabbit or anti mouse IgG as the secondary antibodies (Pharmacia). Anti-MEF2A rabbit polyclonal antiserum (C-21) was from Santa Cruz Biotechnology (Santa Cruz, Calif.). The anti-CD31 (PECAM-1) monoclonal antibody was used as an endothelial-specific marker (Pharmingen-Becton Dickison Co., San Jose, Calif.). Slides were mounted using anti-fading vectashied with DAPI (Vectoris) and cells were viewed under a Zeiss Axioskop fluorescence microscope equipped with photometrics Smartcapture.

Transcription Activation (Luciferase) Assay

HeLa cells were grown to 95% confluence in Dulbecco's minimum essential medium (DMEM) supplemented with 10% fetal bovine serum and transfected with LipofectAMINE 2000 (Invitrogen) and 50 ng of DNA for the expression construct, 1 μg of DNA for the reporter gene, and 50 ng of internal control plasmid pSV—galactosidase. Cells were harvested and lysed 24 h after transfection.

The efficiency of transfection was examined by Western blot analysis. Forty μg of total cellular lysates were separated by 12% SDS-PAGE and electro-transferred to a polyvinylidene fluoride membrane. The membrane was probed with goat polyclonal anti-MEF2A antiserum (Santa Cruz Biotechnology, Santa Cruz, Calif.) as the primary antibody and the rabbit anti-goat IgG horseradish peroxidase-conjugated secondary antibody (Santa Cruz Biotechnology, Santa Cruz, Calif.). ECL Western blotting detection reagents (Amersham Pharmacia Biotech) were used to visualize the protein signal.

Luciferase assay was performed using a Dual-Luciferase assay kit according to the manufacturer's instructions (Promega). The β-galactosidase activity expressed from pSV—galactosidase was used to normalize the transfection efficiency. The experiments were repeated two times in triplicate. Data are expressed as mean±S.E.

TABLE S1
PCR primers for amplification of MEF2A exons.

| | | | |
|---|---|---|---|
| | | | Annealing |
| Exon(s) | Forward Primer (5′ to 3′) | Reverse Primer (5′ to 3′) | Temp. |

| | | | |
|---|---|---|---|
| 1 | AGAAGCTGTGTACGATGCATTAG | ACCCAACCATTCTGTCTATGTT | 64° C. |
| | (SEQ ID NO: 9) | (SEQ ID NO: 10) | |
| 2 | AGATTCATCTTCAGATAGCCCATA | ACAAGTCATTCTGACAGTTAATGC | 64° C. |
| | (SEQ ID NO: 11) | (SEQ ID NO: 12) | |
| 3 | AGTTCATTCCGTCTGTGCTCTCT | AAGTAGAGGTAAAGTAAAAGTACTTA | 66° C. |
| | (SEQ ID NO: 13) | (SEQ ID NO: 14) | |
| 4 | TAAGTACTTTTACTTTACCTCTACTT | GCAACAAGATGTTTGGTCAATCTCT | 66° C. |
| | (SEQ ID NO: 15) | (SEQ ID NO: 16) | |
| 5 | AGTAACTTGAGTTACCTTGCCA | GAACCTGCTTATGTTAACCAATGA | 50° C. |
| | (SEQ ID NO: 17) | (SEQ ID NO: 18) | |
| 6 | TCTCTATT CAGTTCACGT TCAGTTA | TGTATTAGTGAAAGTACCCTTCAG | 50° C. |
| | (SEQ ID NO: 19) | (SEQ ID NO: 20) | |
| 7 | GATACTCAAACCTGTAGTGAGT | GGAAGCTACAGATTGACTATGT | 55° C. |
| | (SEQ ID NO: 21) | (SEQ ID NO: 22) | |
| 8 | TGTGAGTACCAACAGTCTTAGTA | GGTTAGATAACAACACGTAAGAG | 60° C. |
| | (SEQ ID NO: 23) | (SEQ ID NO: 24) | |
| 9 | TCACATCATCAGTGCTTCAGAA | CACAGAAGCACACGTTGATCA | 64° C. |
| | (SEQ ID NO: 25) | (SEQ ID NO: 26) | |
| 10 | ATAGATTCCGTATGGACCTTCCA | AAGACAGTGTGTAGGCCAGGAGTG | 66° C. |
| | (SEQ ID NO: 27) | (SEQ ID NO: 28) | |
| 11 | TGCAGAGGTACTTGCAAGCCAT | AGATATGTAGGGCAGGTCACT | 64° C. |
| | (SEQ ID NO: 29) | (SEQ ID NO: 30) | |

TABLE S2
Known and putative genes located in the adCAD/MI1 locus*.

| ID# | Gene Name | Potential Function |
|---|---|---|
| 1 | LOC350203 | Similar to poly(A)-binding protein 4 (PABP 4) |
| 2 | LOC342149 | Hypothetical protein XP_296681 |
| 3 | LOC123374 | Similar to histone H3 (LOC123374) |
| 4 | LOC342150 | Similar to ST13-like tumor suppressor |
| 5 | FLJ11175 | Hypothetical protein FLJ11175 |
| 6 | LOC342151 | Hypothetical protein XP_296682 |
| 7 | LOC342152 | Hypothetical protein XP_296683 |
| 8 | LOC350204 | Hypothetical protein XP_303877 |
| 9 | LOC253680) | Similar to glioma tumor suppressor candidate |
| | | region gene 2 protein (p60) |
| 10 | LOC204225 | Hypothetical protein XP_118544 |
| 11 | LOC253682 | Hypothetical protein XP_173727 |
| 12 | LOC342254 | Hypothetical protein XP_296739 |
| 13 | LOC342255 | Hypothetical protein XP_296740 |
| 14 | LOC342256 | Hypothetical protein XP_296741 |
| 15 | LOC145820 | Hypothetical protein LOC145820 |
| 16 | LOC350205 | Hypothetical protein XP_303878 |
| 17 | LOC342257 | Hypothetical protein XP_296742 |
| 18 | LOC350206 | Hypothetical protein XP_303879 |
| 19 | LOC342258 | Hypothetical protein XP_296743 |
| 20 | LOC350207 | Hypothetical protein XP_303880 |
| 21 | LOC350208 | Hypothetical protein XP_303881 |
| 22 | LOC145824 | Hypothetical protein XP_085247 |
| 23 | LOC339025 | Hypothetical protein XP_294778 |
| 24 | NTPK3 | Neurotrophic tyrosine kinase receptor, type3 |
| 25 | LOC55829 | Ad-015 protein |
| 26 | MGC14386 | Similar to cyclin E binding protein 1 |
| 27 | CIB1 | Calcium and integrin binding protein 1 |
| | | (calmyrin), DNA-dependent protein kinase |
| | | interacting protein |
| 28 | ABHD2 | Abhydrolase domain containing 2 |
| 29 | FLJ12572 | Hypothetical protein FLJ12572 |
| 30 | PEX11A | Peroxisomal biogenesis factor 11A |
| 31 | RHCG | Rhesus blood group, C glycoprotein |
| 32 | FLJ12484 | Hypothetical protein FLJ12484 |
| 33 | AP3S2 | Adaptor-related protein complex 3, sigma 2 |
| | | subunit |
| 34 | ANPEP | Alany1 (membrane) aminopeptidase |
| | | (aminopeptidase M, microsomal aminopeptidase, |
| | | CD13, p150) |
| 35 | VAPA | VAMP (vesicle-associated membrane protein) |
| 36 | MFGE8 | Milk fat globule EGF factor 8 protein |
| 37 | RLBP1 | Retinaldehyde binding protein 1 |
| 38 | ISG20 | Interferon stimulated gene 20 kDa |
| 39 | FES | Feline sarcoma oncogene |
| 40 | PRO2198 | Hypothetical protein PRO2198 |
| 41 | MGC45866 | Hypothetical protein MGC45866 |
| 42 | POLG | Polymerase (DNA directed) gamma |
| 43 | IR1899308 | Hypothetical protein IR1899308 |
| 44 | PRC1 | Protein regulator of cytokinesis 1 |
| 45 | MRPL46 | Mitochondrial ribosomal protein L46 |
| 46 | MRPS11 | Mitochondrial ribosomal protein S11 |
| 47 | ADAMTS17 | Disintegrin-like and metalloprotease (reprolysin |
| | | type) with thrombospondin type 1 motif, 17. The |
| | | function of this protein has not been determined. |
| 48 | SNRPA1 | Small nuclear ribonucleoprotein polypeptide A |
| 49 | MAN2A2 | Mannosidase alpha, class 2A, member 2 |
| 50 | FLJ12484 | Hypothetical protein FLJ12484 |
| 51 | MGC18216 | Hypothetical protein MGC18216 |
| 52 | FLJ23119 | Hypothetical protein FLJ23119 |
| 53 | RGM | Likely ortholog of chicken repulsive guidance |
| | | molecule |
| 54 | IQGAP1 | IQ motif containing GTPase activating protein 1 |
| | (SAR1) | |
| 55 | IDH2 | Isocirate dehydrogenase 2(NADP+), |
| | | mitochondrial |
| 56 | BLP2 | BBP-like protein 2 |
| 57 | FLJ23119 | Hypothetical protein FLJ23119 |
| 58 | ALDH1A3 | Aldehyde dehydrogenase 1 family, member A3 |
| 59 | DMN | Desmuslin |
| 60 | FLJ35955 | Hypothetical protein FLJ35955 |
| 61 | FLJ10103 | Hypothetical protein FLJ10103 |
| 62 | FLJ12572 | Hypothetical protein FLJ12572 |
| 63 | NR2F2 | Nuclear receptor subfamily 2, group F, member 2 |
| 64 | CHD2 | Chromodomain helicase DNA binding protein 2 |
| 65 | MEF2A | MADS box transcription enhance factor 2, |
| | | polypeptide A. Early marker for cells of |
| | | vasculature |
| 66 | FLJ21868 | Hypothetical protein FLJ21868 |
| 67 | NEUGRIN | Mesenchymal stem cell protein DSC92 |
| 68 | FLJ21868 | Hypothetical protein FLJ21868 |
| 69 | BLM | Bloom syndrome gene |
| 70 | VPS33B | Vacuolar protein sorting 33B |
| 92 | MGC44294 | Hypothetical protein MGC44294 |
| 72 | IROO039700 | Hypothetical protein IROO039700 |
| 73 | FURIN | Furin (paired basic amino acid cleaving enzyme) |
| 74 | FLJ11175 | Hypothetical protein FLJ11175 |
| 75 | FLJ23119 | Hypothetical protein FLJ23119 |
| 76 | MGC24976 | Hypothetical protein MGC24976 |
| 77 | TRA1 | Tumor rejection antigen (gp96) 1 |
| 78 | IROO039700 | Hypothetical protein IROO039700 |
| 79 | FLJ31461 | Hypothetical protein FLJ31461 |
| 80 | AGC1 | Aggrecan 1(chondroitin sulfacte rpoteoglycan 1 |
| | | large aggregation proteoglycan antigen identified |
| | | by monoclonal antibogy A0122) |
| 81 | FLJ12484 | Hypothetical protein FLJ12484 |
| 82 | ASB7 | Ankyrin repeat and SOCS box-containing 7 |
| 84 | SV2B | Synaptic vesicle glycoprotein 2B |
| 85 | CHSY1 | Carbohydrate (chondroitin) synthase 1 |
| 86 | WINS1 | WINS1 mRNA was expressed in adult testis, |
| | | prostate, spleen, thymus, skeletal muscle, fetal |
| | | kidney & brain |
| 87 | IGF1R | Insulin-like growth factor 1 receptor |
| 88 | AP3S2 | Adaptor-related protein complex 3, sigma 2 |
| | | subunit |
| 89 | PLIN | Perilipin |
| 90 | PACE4 | Paired basic amino acid cleaving system 4 |
| 91 | FLJ25005 | Hypothetical protein FLJ25005 |
| 92 | ALDH6 | Aldehyde dehydrogenase 6 |
| 93 | ALDOB | Aldolase B, fructose-bisphosphate |

*Compiled by searching the UCSC Genome Bioinformatics site with UCSC Genome Browser (http://genome.ucsc.edu/index.html?org=Human), the GeneMap99 Database (www.ncbi.nlm.nih.gov/genemap/page.cgi?F=Home.html) and Unigene Database at NCBI (www.ncbi.nlm.nih.gov/entrez/query.fcgi?db=unigene).

### Example 2

### Identification of Three Novel Mutations in MEF2A Associated with Coronary Artery Disease and Myocardial Infarction

Here we report the results from mutational analysis of MEF2A in 200 independent patients with CAD/MI (males<55 years and females<60 years of age) and 200 individuals with normal angiograms.

Methods

Mutational analysis was carried out using single strand conformation polymorphism (SSCP) and DNA sequence analyses. The functional consequence of the newly-identified MEF2A mutations were examined using a transcription activation assay with the ANF₋₇₀₀p-luciferase reporter gene and transient transfection of wild type or mutant MEF2A proteins in HeLa cells. The results are shown in FIG. 9.

Results

The three novel mutations were identified in exon 7 of MEF2A in four of 200 CAD/MI patients (2%), and none of the mutations were detected in 200 normal individuals. These mutations include N263S identified in two independent CAD/MI patients, P279L in one patient, and G283D in another patient (FIGS. 7, 8, and 6, respectively). Analysis of family members revealed that the father of the patient with mutation P279L also carried the mutation and has the diagnosis of CAD. The three mutations are located within or close to the major transcription activation domain of MEF2A (amino acids 274-373), and significantly reduced the transcription activation activity of MEF2A. These results suggest that N263S, P279L, and G283D are functional mutations.

Conclusions

These results provide the first confirmatory evidence of our previous report that MEF2A mutations cause CAD and MI and indicate that a significant percent of the CAD/MI population (2%) may carry mutations in MEF2A. Further definition of the prevalence of MEF2A mutations is clearly warranted.

### Example 3

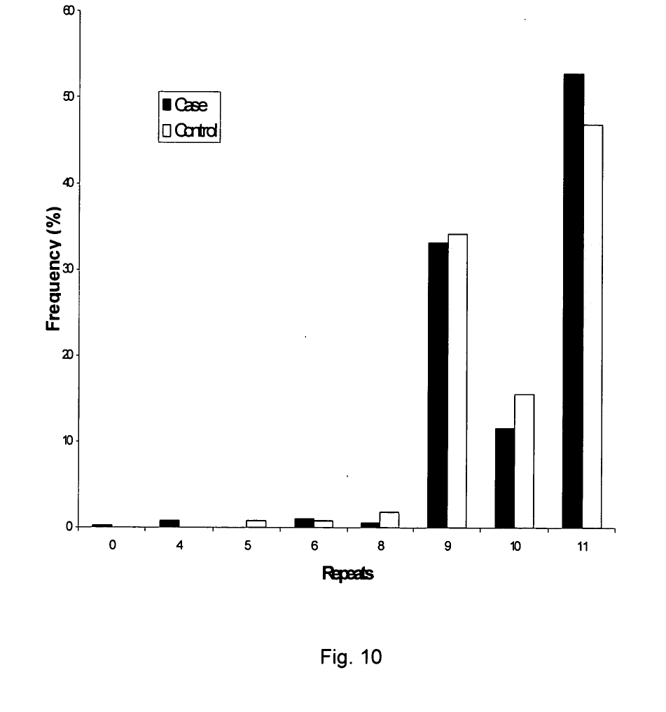

To test if the CAG repeat in MEF2A is a significant genetic factor for premature myocardial infarction, 190 cases and 199 controls were randomly selected from GeneQuest and the general populations, to be genotyped. The distributions of CAG repeats within cases and controls are shown in FIG. 10. A logistic regression assuming a logit relationship between the phenotype and the underlying molecular determinant (the cumulative repeats contained in the genotype) was used to assess the effects of the CAG repeat. The analysis results demonstrate a slight decrease of the log of disease risk odds (−0.04) for each repeat increase but not up to a statistical significance (P=0.53), suggesting that this repeat may acts as a neutral site as a conventional microsatellite in non-coding regions does.

Having described the invention the following is claimed:

1. A method of identifying a person at risk of developing coronary artery disease:
detecting an alteration of at least one of an MEF2A gene, genes regulated by MEF2A transcription factor, or genes that regulate expression of MEF2A transcription factor of the person, the alteration substantially reducing the transcription activity of the resulting MEF2A transcription factor.

2. The method claim 1, the alteration in the MEF2 disrupting the nuclear localization of the MEF2A transcription factor.

3. The method of claim 1, the alteration comprising a mutation in the coding region of the MEF2A gene, the mutation impairing transcription activity of the MEF2A protein.

4. The method of claim 3, the mutation resulting in at least one of a insertion, deletion, point mutation, or inversion of nucleic acids in at least one of exon 7 or exon 11 of the MEF2A gene.

5. The method of claim 4, the mutation of the MEF2A gene resulting in deletion of amino acids 440-446 of a wild type MEF2A protein corresponding to SEQ ID NO: 2.

6. The method of claim 4, the mutation of the MEF2A gene resulting in the deletion of at least 5 of the contiguous glutamines of amino acids 420-430 of a wild type MEF2A protein corresponding to SEQ ID NO:2.

7. The method of claim 4, the mutation resulting in at least one of a proline to leucine substitution at amino acid 279, a asparagine to serine substitution at amino acid 263, or a glycine to aspartic acid substitution at amino acid 283.

8. The method of claim 1, detection of the alteration being performed by amplifying at least one of exons 1, 2, 3, 4, 5, 6, 7, 8, 9, 10, and 11 of the MEF2A gene by polymerase chain reaction and analyzing the amplification products produced by the polymerase chain reaction for mutations.

9. The method of claim 8 the analyzing of the amplification products being performed by heteroduplex or single strand conformation polymorphism analysis.

10. A method of diagnosing an individual who has coronary artery disease or a predisposition for coronary artery disease:
providing a nucleic acid sample from the individual, the nucleic acid comprising nucleic acid sequence corresponding to at least one of an MEF2A gene, genes regulated by MEF2A transcription factor, or genes that regulate expression of MEF2A transcription factor;
determining if the nucliec acid sequence corresponding to at least one of an MEF2A gene, genes regulated by MEF2A transcription factor, or genes that regulate expression of MEF2A transcription factor of the patient are mutated such that the mutation impairs the transcription activation activity of a resulting MEF2A transcription factor.

11. The method claim 10, the mutatio disrupting the nuclear localization of the MEF2A transcription factor.

12. The method of claim 10, the mutation resulting in at least one of a insertion, deletion, point mutation, or inversion of nucleic acids of SEQ ID NO: 1.

13. The method of claim 10, the mutation resulting in deletion of amino acids 440-446 of a wild type MEF2A protein corresponding to SEQ ID NO: 2.

14. The method of claim 10, the mutation of the MEF2A gene resulting in the deletion of at least 5 of the contiguous glutamines of amino acids 420-430 of a wild type MEF2A protein corresponding to SEQ ID NO:2.

15. The method of claim 14, the mutation resulting in at least one of a proline to leucine substitution at amino acid 279, a asparagine to serine substitution at amino acid 263, or a glycine to aspartic acid substitution at amino acid 283.

16. The method of claim 10, detection of the mutation being performed by amplifying at least one of exons 1, 2, 3, 4, 5, 6, 7, 8, 9, 10, and 11 of the MEF2A gene by polymerase chain reaction and analyzing the amplification products produced by the polymerase chain reaction for mutations.

17. The method of claim 16, the analyzing of the amplification products being performed by heteroduplex or single strand conformation polymorphism analysis.

18. A method of diagnosing an individual who has coronary artery disease or a predisposition for coronary artery disease:
detecting mutation of the amino acid sequence the MEF2A protein encoded by the MEF2A gene, the alteration substantially reducing the transcription activity of the resulting MEF2A protein.

19. The method of claim 18, the mutation comprising at least one of a insertion, deletion, point mutation, or inversion of the amino acid sequence of a wild type MEF2A protein.

20. The method of claim 19, the mutation comprising a deletion of amino acids 440-446 of the wild type MEF2A protein.

21. The method of claim 18, the mutation of the MEF2A gene resulting in the deletion of at least 5 of the contiguous glutamines of amino acids of the wild type MEF2A protein.

22. The method of claim 18, the mutation resulting in at least one of a proline to leucine substitution at amino acid 279, a asparagine to serine substitution at amino acid 263, or a glycine to aspartic acid substitution at amino acid 283.
